(19) United States
(12) Patent Application Publication
(10) Pub. No.: US 2026/0083503 A1
(43) Pub. Date: Mar. 26, 2026

(54) MACHINE AND PROCESS FOR INSERTING A PROBE IN A BRAIN

(71) Applicant: Board of Regents, The University of Texas System, Austin, TX (US)

(72) Inventors: Nitin Tandon, Houston, TX (US); Mehmet Cihan Kadipasaoglu, Houston, TX (US)

(21) Appl. No.: 18/891,981

(22) Filed: Sep. 20, 2024

(51) Int. Cl. A61B 34/10; A61B 34/20; A61B 90/00

(52) U.S. Cl. CPC A61B 34/10; A61B 34/20; A61B 90/03; A61B 2034/102; A61B 2034/105; A61B 2034/107; A61B 2090/034

(57) ABSTRACT

A machine and process for automatically determining the optimal dimensions and configurations of probes to be placed in the brain for either recording brain activity or for the delivery of electrical current, chemicals, liquids or cells. This system uses three dimensional stereotactic planning and provides an optimization for hardware to be implanted based both on available manufactured devices as well as devices that may possibly need to be manufactured specifically for individual use. Systems for determining the thickness of the skull bone to be removed and for the optimal anchoring devices placed to hold such probes and electrodes temporarily in place are also described. A real time system for detecting errors based on prior calculated trajectory lengths versus lengths actually implanted is also implemented.

### BACKGROUND INFORMATION

### 1. Field

The present disclosure relates generally to a biomedical system and, in particular, to a machine and/or process for selecting and/or designing a probe to place in a brain.

### 2. Background

Abnormal electro-chemical nervous signals generated in a brain of an individual may lead to disorders of a central nervous system of the individual. In some cases, such as without limitation epilepsy, the brain may cause, without limitation, a tendency towards recurrent, unprovoked seizures, episodes of abnormal electrical activity of the central nervous system. These conditions may intermittently incapacitate the individual or in some case may result in convulsions.

The individual may have a condition that is not determinable and/or treatable using only non-invasive brain recordings or evaluations. Hence, the individual may require implantation of a probe into their brain, without limitation for the evaluation of: neurological diseases such as epilepsy, movement disorders, speech or memory or hearing impairment, and/or psychiatric illnesses.

Examination and/or treatments may include intracranial electrodes implanted in the brain that may record high spatiotemporal resolution intracranial electroencephalography (icEEG) data. Examination and/or treatments may include an intracranial electrode modulating a neural circuit and/or system in the individual. An electrode may also be placed for neuromodulation of epilepsy - currently that may be at least in a nucleus of the thalamus or within distinct cortical or subcortical sites of the genesis of seizures. Precise placement of the electrode may be critical for evaluation and/or treatment of various neurological conditions.

Some disorders of action, including but not limited to speech or movement, from brain injury or degeneration may be reduced. Therefore, it would be desirable to have a process and/or machine to record brain activities as icEEG signals and then feed the icEEG signals into another machine to decode brain processes.

It would be desirable to have a process and/or machine that optimize the placement of an electrode into a brain, and in particular, to more effectively determine an optimal type and/or dimensions of a probe for the placement and recordings of the electrode within the brain.

Some disorders of cognition or perception including but not limited to hearing, vision, or memory from brain injury or degeneration may be reduced.

Therefore, it would be desirable to have a process and/or machine to record brain activities as icEEG signals and then feed the icEEG signals into another machine to decode brain processes. It would be desirable to have a process and/or machine that optimized the placement of an electrode into a brain, and in particular, to more effectively determining an optimal type and/or dimensions of a probe for the placement and recordings of the electrode within the brain.

It appears that some psychiatric disorders including (but not limited to) major depressive disorder, anxiety, post-traumatic stress disorder, obsessive compulsive disorder, and/or anorexia, may be reduced using neuromodulation. Therefore, it would be desirable to have a process and/or machine that take into account at least some of the issues discussed above, as well as other possible issues. For example, it would be desirable to have a process and/or machine that overcome a technical problem with risks involved in placing an electrode into a brain, and in particular, to more effectively determine an optimal probe for the placement and operation of the electrode within the brain.

Some disorders of genetic, molecular, cellular, or systems level neurobiology, including but not limited to metabolic, neoplastic or metaplastic processes, may be reduced from inborn or acquired conditions. Therefore, it would be desirable to have a process and/or machine that optimizes the placement of a probe into a brain or said brain's surrounding structures, and in particular, to more effectively determine an optimal type and/or dimensions of a probe for the sampling of tissue or delivery of therapeutics within the brain.

Some disorders of cerebrospinal fluid (CSF) homeostasis may be reduced from congenital or acquired conditions ventriculomegaly. Therefore, it would be desirable to have a process and/or machine that optimizes the placement of a probe into a brain or said brain's surrounding or internal fluid spaces or containing structures, and in particular, to more effectively determine an optimal type and/or dimensions of a probe for the management of CSF or other fluids within the brain.

### SUMMARY

An aspect of the present disclosure provides a process for selecting and/or designing an optimum probe for inserting into a brain.

Yet another aspect of the present disclosure provides a machine configured to select an optimum probe to be implanted in a brain, wherein the machine comprises a processor and a non-transitory computer-readable medium that comprises instructions stored thereon and configured to, when executed by a computer system, direct the computer system to receive: an entry point; a target; a target reference length; a length of a fixation device; a fixation device depth indication; and an inventory of probes. The instructions may also be configured to derive: a working length; and a mismatch between the working length and a proposed working length, respectively, of each probe in the inventory; and select the optimum probe from the inventory. The processor may be further configured to communicate with a second processor in a neuronavigational system that comprises an alignment mechanism.

Yet another aspect of the present disclosure provides a machine that may be configured to design an optimum probe to be implanted in a brain, wherein the machine may include a Probe-Select configured to: select an entry point and a target within the brain; connect a neuronavigational system to a skull; select dimensions for a fixation device; generate a real-time registration of the brain; co-register the neuronavigational system between an anatomical model of the brain and a pre-op model of the brain; determine in real time a working length for the optimum probe, wherein a distance from a deep end of the fixation device to the target within the brain defines the working length; and create, based upon: the working length, the targets, and the entry point, a custom design for the optimum probe that designates: a recording depth, a number of functional elements, a width for each respective functional element in the number of functional elements, and a spacing respectively between adjacent functional elements, along the optimum probe.

The machine may further include the neuronavigational system configured to determine a length from a top of an alignment mechanism of the neuronavigational system to the target.

A depth stop of the machine may be set flush at a top of the alignment mechanism connected to the fixation device secured in the skull. The Probe-Select may be further configured to derive targets that include the target in the brain for optimal implantation of the number of functional elements configured to modulate an anatomical model of the brain. The Probe-Select may also be configured to receive a distance from a depth stop on a driver, used to a tip of the driver connected to the fixation device within the skull. The Probe-Select may also be configured to receive a number of targets, wherein the number of electrodes are located on the optimum probe to modulate the number of targets.

### BRIEF DESCRIPTION OF THE DRAWINGS

The novel features believed characteristic of the illustrative examples are set forth in the appended claims. The illustrative examples, however, as well as a preferred mode of use, further objectives and features thereof, will best be understood by reference to the following detailed description of an illustrative example of the present disclosure when read in conjunction with the accompanying drawings, wherein:

FIG. 1 is an illustration of a cross-sectional side-view of a portion of a brain in accordance with an illustrative example;

FIG. 2 is an illustration of a cross-sectional side-view of a portion of a brain with a probe inserted therein in accordance with an illustrative example;

FIG. 3 is an illustration of a side-view of a bushing of a stereotactic navigation system positioned to contact a scalp shown in a cross-sectional side-view in accordance with an illustrative example;

FIG. 4 is an illustration of a perspective view of a stereotactic navigation system connected to a brain in accordance with an illustrative example;

FIG. 5 is an illustration of a cross-sectional side-view of a drill in a bushing of a stereotactic navigation system positioned to contact a scalp in accordance with an illustrative example;

FIG. 6 is an illustration of a cross-sectional side-view of a driver in a bushing of a stereotactic navigation system and an anchor bolt anchored through an opening through a skull in accordance with an illustrative example;

FIG. 7A is an illustration of a cross-sectional side-view of a portion of a brain with a probe inserted therein and connected to an anchor bolt in accordance with an illustrative example;

FIG. 7B is an illustration of a cross-sectional side-view of a probe with electrodes for insertion into a brain in accordance with an illustrative example;

FIG. 8A is a block diagram of a machine and a process that selects an optimum probe for insertion into a brain in accordance with an illustrative example;

FIG. 8B is a block diagram of a machine and a process that selects an optimum probe for insertion into a brain in accordance with an illustrative example;

FIG. 9 is an illustration of an example of an inventory of probes available for implanting into a brain in accordance with an illustrative example;

FIG. 10 is an illustration of a flowchart of a probe manufacturing and service process depicted in accordance with an illustrative embodiment;

FIG. 11 is an illustration of a block diagram for a probe in accordance with an illustrative embodiment;

FIG. 12 is an illustration of a block diagram of a data processing system in accordance with an illustrative example; and

FIG. 13 is an illustration of a flowchart of a process for selecting a probe for inserting into a brain in accordance with an illustrative example.

### DETAILED DESCRIPTION

The illustrative examples recognize and take into account one or more different considerations. For example, the illustrative examples recognize and take into account that non-invasive studies or evaluations of activity in a brain may include without limitation, electroencephalography (EEG), magnetoencephalography (MEG), as well as anatomical imaging modalities used to identify at least structural lesions such as magnetic resonance imaging (MRI) or computed tomography (CT).

Further, the illustrative examples recognize and take into account that movement disorders (such as without limitation, Parkinson's disease, dystonia, essential tremor) may be common disorders from efficient brain function. The treatment of these disorders may often be surgical, as medications generally result in undesirable side effects. Deep basal ganglia nuclei (e.g., subthalamic nucleus, globus pallidus interna and the VIM nucleus of the thalamus) and their associated white matter pathways are routinely targeted for treatment to minimize effects of these disorders. Additionally, psychiatric disorders, such as without limitation: resistant depression, obsessive compulsive disorder, post-traumatic stress disorders and eating disorders may be treated by neuromodulation.

The illustrative examples recognize and take into account that psychiatric disorders may be reduced from neuromodulation. Psychiatric disorders that may be reduced from neuromodulation may include without limitation: medication resistant depression, obsessive compulsive disorder, post-traumatic stress disorders and eating disorders.

The illustrative examples recognize and take into account that inborn or acquired disorders of neurobiology may be reduced from targeted delivery of therapeutics. Inborn or acquired disorders that may be reduced from targeted therapeutics may include without limitation: genetic, metabolic, autoimmune, inflammatory disorders and primary or secondary/metastatic neoplasms.

The illustrative examples recognize and take into account that inborn or acquired disorders of cerebrospinal fluid (CSF) homeostasis may be reduced modulation of abnormal fluid contents by a probe or catheter. CSF disorders that may be reduced from modulation of abnormal fluid contents by a probe or catheter include without limitation: internal and external congenital or acquired hydrocephalus, including communicating, obstructive, and normal pressure hydrocephalus.

The illustrative examples recognize and take into account that implantation of stereo-electroencephalography (SEEG) electrodes and/or other depth electrodes or other recording machines may be a strategy used to diagnose and/or to stimulate and cause neuromodulation. Herein, an electrode refers to an element that may receive or transmit electronic signals. One or more electrodes may be located along or within a probe. An electrodes may be powered through a probe. Herein a probe refers to an element that may be inserted into a brain. Accordingly, a probe placed into a brain may be used to precisely define a relationship between a healthy and/or eloquent brain region and a pathologic brain region that may underlie a putative pathological network. Thus, where the term probe is used herein, one of ordinary skill in the art recognizes that probe may be representative of an equivalent implantable device such as without limitation a catheter.

Some disorders of action, including but not limited to: speech or movement, or of perception such as vision or sound or sensation or smell, or of cognition such as memory or reasoning from brain injury or degeneration may be reduced. Therefore, it would be desirable to have a process and/or machine or device to record brain activities as icEEG signals in a computer or similar machine to read, analyze, and thereby derive meaning from or decode brain processes. In another instantiation, the administration of controlled electrical currents into the brain may recreate lost or impaired sensation or cognitive ability. It would be desirable to have a process and/or machine that optimizes the placement of an electrode into a brain, and in particular, to more effectively determine the optimal type and dimensions of a probe for the placement and recordings of the electrode within the brain.

The illustrative examples recognize and take into account that implantation of subdural electrodes (SDEs) in patients with pharmaco-resistant epilepsy is a common strategy in epilepsy surgery that is employed when there is no discrete electro-physiologically abnormal lesion. To properly plan the cortical resection, precise electrode locations are generally needed to determine the proximity of the seizure focus relative to well-established anatomic or functional landmarks. Beyond this, accurate electrode localization is generally crucial to the process of integrating intracranial electrophysiological data with other imaging techniques used to map out the epileptogenic network, such as without limitation: computed tomography (CT) imaging, magnetic resonance imaging (MRI), localization of inter-ictal spikes by magneto-encephalography (MEG), measurements of aberrant cerebral blood flow and metabolism with single photon emission computed tomography (SPECT) or positron emission tomography (PET). Furthermore, accurate electrode localization allows for the identification of eloquent cortical regions, and functional localization data, principally from functional magnetic resonance imaging (fMRI) and MEG recordings overlaid onto the pre-surgical MRI scans. One of ordinary skill in the art recognizes that these data sets are essential prior to some resections and need to be precisely co-registered (aligned) onto the individual patient's brain volume for comparison with the findings from the SDE recordings. In addition, there is a growing interest in the use of SDE-based intracranial electro-corticographic (ECoG) data to provide unique insights into cerebral networks involved in motor function, language, and cognitive control. Individual and grouped analyses of these data rely on precise electrode localization due to the co-registration of data between subjects.

Stereo-electroencephalography (SEEG), may involve placement of multiple (in some instances 8-16) probes into myriad cortical and limbic targets using frameless and/or frame-based stereotactic navigation systems that allow for the use of orthogonal or vertical or longitudinal or azimuth-based trajectories to guide probe placement. See at least: U.S. Pat. No. 10,589,096 B2 issued Mar. 17, 2020, entitled “Systems and Methods for Network Based Neurostimulation of Cognitive Processes” and incorporated herein in its entirety; U.S. Patent Application Publication 2021/0264623 A1 published Aug. 26, 2021, entitled “Methods for Optimizing the Planning and Placement of Probes in the Brain via Multimodal 3D Analysis of Cerebral Anatomy” and incorporated herein in its entirety; U.S. Pat. No. 11,497,401 issued Nov. 15, 2022, entitled “Methods for Localization and Visualization of Electrodes and Probes in the Brain Using Anatomical Mesh Models” and incorporated herein in its entirety. Such multi-directional trajectories allow for probe placement into any cortical structure.

When appropriate, placement of a probe into a brain may be broadly classified into three stages: 1) surgical planning for the identification of cranial or intracranial regions of interest (ROI), including extra-axial regions comprising epidural, subdural, sub-arachnoid, or sub-pial spaces; vascular or intravascular ROIs, as well as in intra-axial ROIs comprising cortical or subcortical ROIS, themselves which may further comprise at least a thalamic, basal ganglia, cerebellar, or brain stem anatomy of a brain (heretofore collectively referred to as ROIs) from which data should be sampled; 2) determining the optimal trajectory for each ROI that a given probe implanted into the brain should traverse (e.g. from the patient's scalp to the target within the cortical area); and 3) determining an optimal probe for each trajectory (e.g. based on the trajectory length as well as probe specifications).

Hence, the illustrative examples herein recognize and take into account that at the current time there is a need for a novel process and/or machine that provides effective strategies for determining the optimal probe to be placed into the brain at the time of surgery. One of ordinary skill in the art recognizes that to date, a professional opinion approach, using individual medical personnel experience and approximations rather than patient-specific anatomic-physiological boundaries, has been used by surgeons to select a particular type and/or dimensions and electrode configurations of each probe that is implanted.

In contrast, the novel technological improvement presented by the illustrative examples herein recognize and describe a semi-automated process and/or a machine that integrates precise navigational metrics provided by stereotactic neurosurgical robotic systems with anatomical information for a specific individual patient using stereotactic measurements taken intra-operatively and probe-specific geometric constraints. Thereby, illustrative examples herein recognize and describe a sophisticated, synergistic process and/or machine for selecting an optimal probe and the electrode distribution along the probe for a given trajectory into the cortical structure. The process and/or machine execute in real-time, thus providing the technological improvement of reducing the time required by the surgeon to implant a probe.

One of ordinary skill in the art recognizes that while the discussion and examples herein illustrate precise electrode placement along a probe, and hence at a particular location within a brain, that key components on a probe other than an electrode may take the place of the electrode in discussions herein. As a nonlimiting example, in place of an electrode, a micro applicator or releasable capsule may be positioned along a probe. A coring device on the probe for biopsies may also be considered in place of an electrode on a probe as discussed herein. Such key components may be combined on a single probe and may be referred to generally as functional elements located on the probe. In other words, electrodes on a probe may be referred to as functional elements on/of the probe.

The novel process and/or machine presented by illustrative examples herein provide the technological improvement at least of reducing the potential for error introduction when selecting a specific probe and placing the selected probe into the brain - as compared to selecting a specific probe and placing the selected probe into the brain without using the novel Probe-Select and/or associated process. The process and/or machine execute probe selection for implant in a brain in real-time, and thus may reduce without limitation—as compared to selecting a specific probe and placing the selected probe into the brain without using the novel Probe-Select and/or associated process—time required for a surgical team to derive selection of an optimal probe and thereby reducing actual time under anesthesia for a patient and thus reducing peri-operative complications that may result from extended time under anesthesia. The novel Probe-Select and/or associated process execute probe selection for implant in a brain in real-time, and thus may reduce without limitation - as compared to selecting a specific probe and placing the selected probe into the brain without using the novel Probe-Select and/or associated process: personnel required for the operation and/or additional risks therefrom including without limitation: distraction, obstruction, error, and/or infection; and/or an infection risk proportional to a duration of time that the surgery requires.

The illustrative examples recognize and take into account that the term “couple” or “couples” may indicate either an indirect or direct wired or wireless connection. Without limitation, if a first machine couples to a second machine, that connection may be through a direct connection or through an indirect connection via other machines and connections.

Further, the recitation “based on” may mean “based at least in part on. ” Therefore, if R is based on S, R may be a function of S and any number of other factors.

As used herein, the phrase “at least one of”, when used with a list of items, means different combinations of one or more of the listed items may be used, and only one of each item in the list may be needed. In other words, “at least one of” means any combination of items and number of items may be used from the list, but not all of the items in the list are required. The item may be a particular object, a thing, or a category.

For example, without limitation, “at least one of item A, item B, or item C” may include item A, item A and item B, or item B. This example also may include item A, item B, and item C or item B and item C. Of course, any combinations of these items may be present. In some illustrative examples, “at least one of” may be, for example, without limitation, two of item A, one of item B, and ten of item C; four of item B and seven of item C; or other suitable combinations.

The illustrative examples recognize and take account that a physician or surgeon or equivalent implanting a probe into a brain must simultaneously monitor the patient, the patient's vitals, the patient's response, direct and monitor the location of embedded electrodes in the brain, actions of the surgical team, as well taking into account as other factors. These examples recognize and take into account that these actions require large amounts of concentration and focus by the implanting individual.

The illustrative examples recognize and take account that it would be useful to have an improved user interface allowing a physician or surgeon or equivalent to minimize infection risk to the brain and to more accurately place electrodes in the brain by more expeditiously and accurately selecting an optimal probe after an anchor bolt is anchored in the brain and more accurately placing the selected probe into the brain.

In one illustrative example, information about an operation of a group of electrodes in the brain of a patient for deep brain stimulation is received. The stimulation information is received in real time during operation of the group of electrodes. The process displays the group of electrodes on a head of a patient on the display system such that the group of electrodes is displayed overlaid on a view of the head of the patient in real time in a position corresponding to the actual position of the group of electrodes in a brain in the head of the patient.

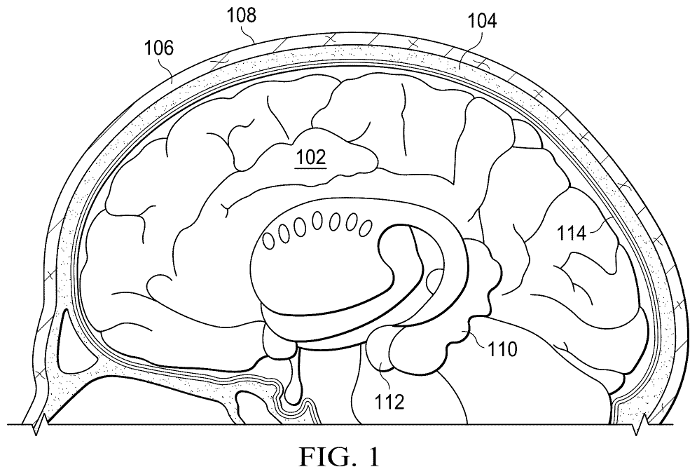

With reference now to the figures, and in particular, with reference to FIG. 1, is an illustration of a cross-sectional side-view of a portion of a brain in accordance with an illustrative example. As introduced above, it may be desirable to place a probe into particular locations within brain 102 within skull 104. Skull 104 may be located some distance beneath surface 108 of scalp 106. Dura mater 114 may be located some distance beneath and within skull 104. Without limitation, hippocampus 110 and/or amygdala 112 may be portions of brain 102 where it would be desirable to place the probe.

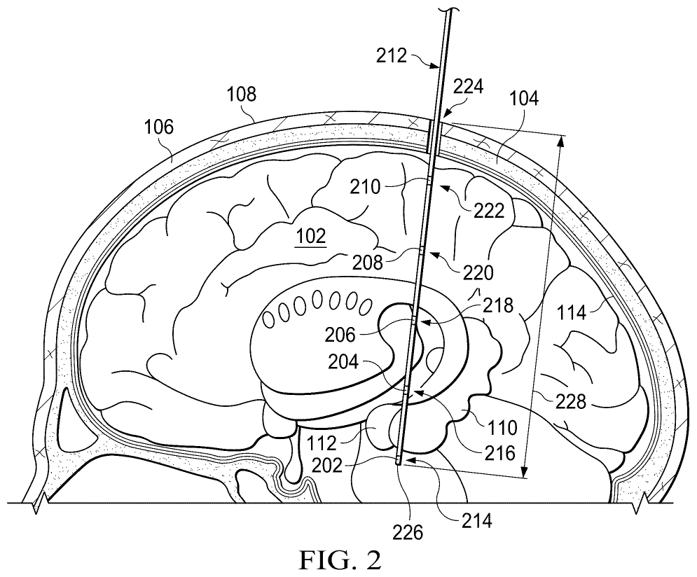

With reference now to FIG. 2, FIG. 2 is an illustration of a cross-sectional side-view of a portion of a brain with a probe inserted therein in accordance with an illustrative example. Specifically, it may be determined that it is desirable to place without limitation: electrode 202, electrode 204, electrode 206, electrode 208, and electrode 210, at one and/or several locations in brain 102. Without limitation, any and/or all of electrode 202, electrode 204, electrode 206, electrode 208, and electrode 210 may be fixed onto probe 212 and inserted in brain 102 to place electrode 202, electrode 204, electrode 206, electrode 208, and/or electrode 210, precisely at the desired locations shown relative to without limitation, hippocampus 110, amygdala 112, skull 104, and/or other portions of brain 102.

Without limitation, the desired location may be any and/or all of the desired locations for target 214, target 216, target 218, target 220, and/or target 222.

Probe 212 may have a number electrodes on it. Without limitation, the number of electrodes on probe 212 may be 8, 10, 12, 14, 16, or some other number.

One of ordinary skill in the art recognizes that FIG. 2 illustrates just one non-limiting example, and that for at least reasons discussed above, other target areas and thus other trajectories for a probe and/or probes in place of or in addition to probe 212 shown in FIG. 2 may be desirable. A trajectory for a probe such as without limitation, probe 212 may be defined by entry point 224 and tip 226 of probe 212 and distance 228 therebetween.

Hence, a machine and/or process are needed to select probe 212 with appropriate dimensions and electrode distribution to locate electrode 202, electrode 204, electrode 206, electrode 208, and electrode 210, precisely at the desired locations for target 214, target 216, target 218, target 220, target 222 shown relative to without limitation, hippocampus 110, amygdala 112, and/or skull 104. Further, one of ordinary skill in the art recognizes that a selection of particular features desired for probe 212 may also affect selection of a particular probe to serve as probe 212. In addition to differing placement of electrodes on probe 212, without limitation, a diameter, a material, electric conductivity features, a stiffness, and/or a particular manufacturer, a particular functionality, of probe 212 may be desirable. Various factors that may influence the selection and/or manufacture of a particular probe to serve as probe 212 may include without limitation, a function performed by the probe and/or electrodes thereon. Likewise, a particular probe may be selected based upon without limitation, dimensions and/or characteristics of each electrode on the particular probe.

One of ordinary skill in the art understands that FIG. 2 merely provides one illustration of potential desired locations and numbers of electrodes for placement within brain 102. Without limitation, probe 212 may have a single electrode or may have numerous electrodes. Without limitation, more probes than just probe 212 may be placed in brain 102 simultaneously. The targeted locations (without limitation target 214, target 216, target 218, target 220, and/or target 222, as shown in FIG. 2) for placement of electrodes (without limitation electrode 202, electrode 204, electrode 206, electrode 208, and electrode 210, as shown in FIG. 2) may be initially decided before placing probe 212 into brain 102 by without limitation at least a machine and/or process as described by U.S. Patent Application Publication 2021/0264623 A1 published Aug. 26, 2021, entitled “Methods for Optimizing the Planning and Placement of Probes in the Brain via Multimodal 3D Analysis of Cerebral Anatomy,” incorporated herein in its entirety. Ideally probe 212 inserted into brain 102 would only have electrodes located at exactly the right lengths from tip 226 to align exactly with any desired targets in brain 102.

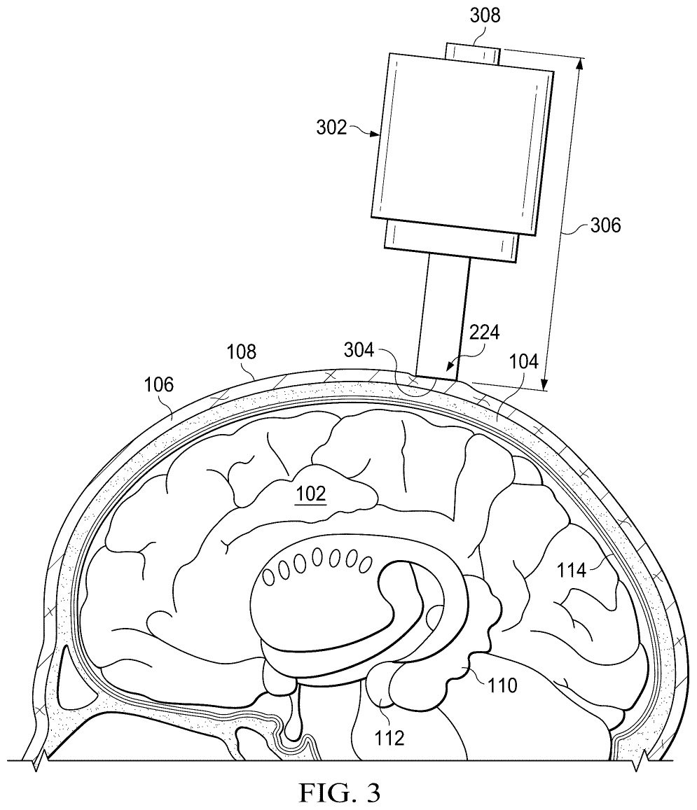

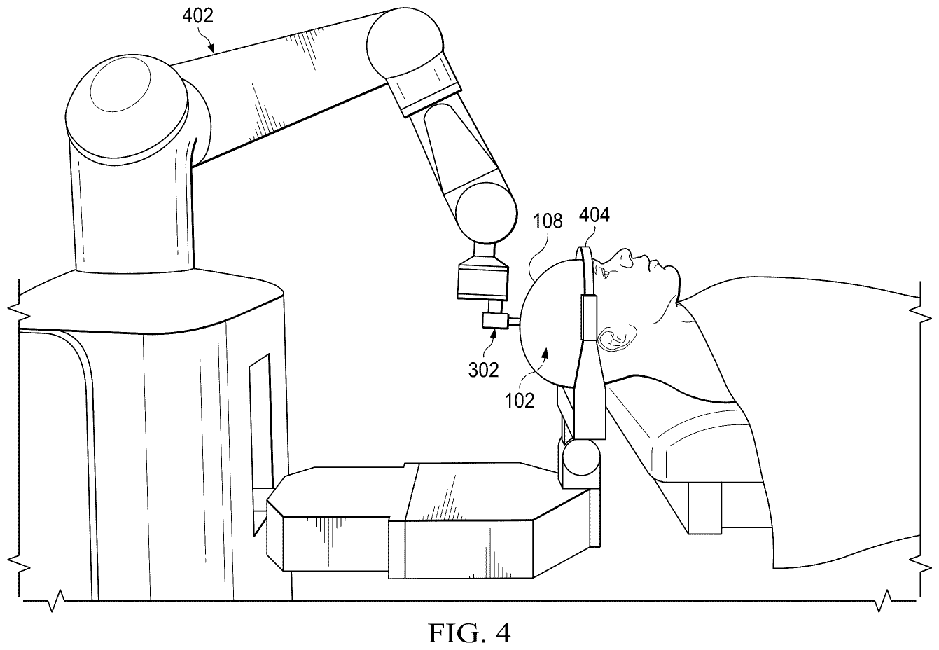

With reference now to FIG. 3, FIG. 3 is an illustration of a side-view of a bushing of a stereotactic navigation system positioned to contact a scalp shown in a cross-sectional side-view in accordance with an illustrative example. More specifically, FIG. 3 shows bushing 302 which is a part of a stereotactic navigation system (see FIG. 4). A bottom 304 of bushing 302 contacts surface 108 of scalp 106 such that a hollow core within the bushing centers around entry point 224 on surface 108 of scalp 106. Hence, a distance 306 exists between entry point 224 of scalp 106 and top 308 of bushing 302.

Entry point 224 may alternatively be considered as representing a point located on applicable related anatomical structures, such as without limitation upon a skull or equivalent surface. Bushing 302 illustrated is representative of an equivalent alignment mechanism for a stereotactic navigation system. In other words, one of ordinary skill in the art recognizes that alignment and contact of a stereotactic navigation system for placing probe 212 into an anatomical structure may be an alternative alignment mechanism functionally equivalent to illustrated bushing 302.

With reference now to FIG. 4, FIG. 4 is an illustration of a perspective view of a stereotactic navigation system connected to a brain in accordance with an illustrative example. As described above, a machine and/or process are needed to be able to select, guide, and precisely implant probe 212 with appropriate dimensions and electrode distribution into brain 102. Without limitation, neuronavigational system 402 may be connected to brain 102 and bushing 302 placed against entry point 224 on surface 108 of scalp 106 as shown in FIG. 3. Neuronavigational system 402 may be and/or incorporate a stereotactic navigation system. Neuronavigational system 402 may be without limitation a neuronavigational system 402 that may be robotic and/or neuronavigational system 402 that may be computerized and/or autonomous in some other manner.

As shown, neuronavigational system 402 may be connected to brain 102 in a manner that co-registers a position of neuronavigational system 402 and parts thereof, such as without limitation bushing 302, to positions of portions within brain 102, such as without limitation, hippocampus 110 and/or amygdala 112, and/or more specifically target 214, target 216, target 218, target 220, and/or target 222. Neuronavigational system 402 may be connected to brain 102 through frame 404 of neuronavigational system 402. As will be discussed further below, without limitation, positions within brain 102 may be identified in an x, y, z coordinate system that is co-registered with an x, y, z coordinate system of neuronavigational system 402.

As used herein, neuronavigational system 402 may refer to any equivalent device configured and capable to register an anatomical structure or structures of interest, such as without limitation: scalp 106, skull 104, brain 102, and/or other equivalent or associated intracranial structures (heretofore referred to as anatomical structures).

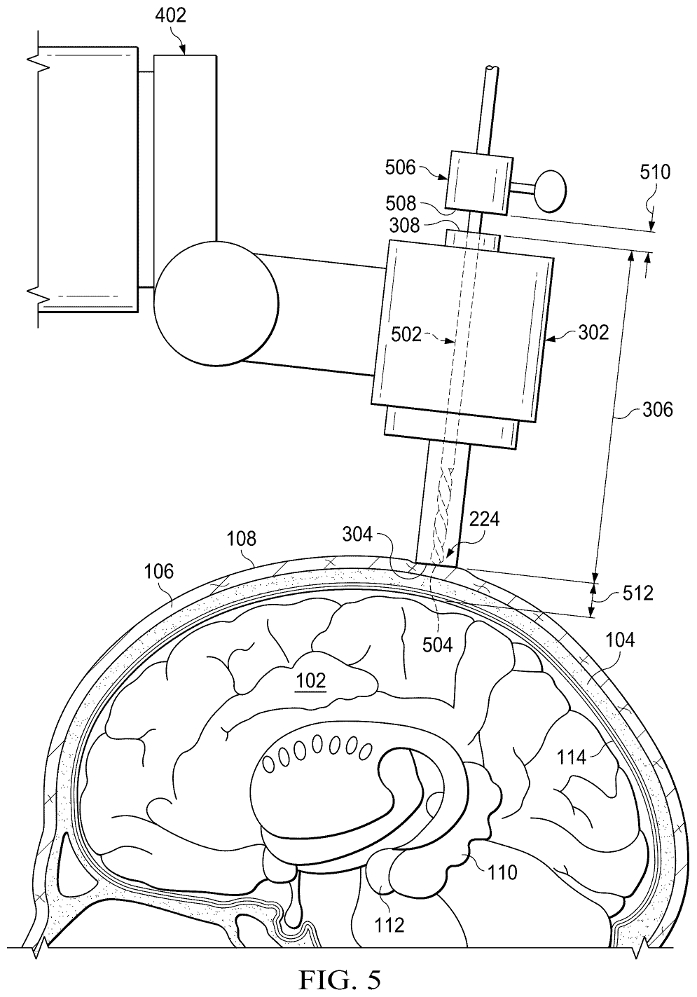

With reference now to FIG. 5, FIG. 5 is an illustration of a cross-sectional side-view of a drill in a bushing of a stereotactic navigation system positioned to contact a scalp in accordance with an illustrative example. Specifically, drill 502 is fed through a central hollow core (not shown) of bushing 302. Without limitation, drill 502 may have a diameter of 2.1 or 2.4 millimeters. Without limitation, a reducing tube (not shown) may be placed within the central hollow core of bushing 302. As a non-limiting example, when diameter of drill 502 is 2.1 millimeters, a reducing tube (not shown) may be placed within the central hollow core of bushing 302. The reducing tube may function to keep drill 502 centered on entry point 224 beneath center of the central hollow core of bushing 302.

To prepare for placing probe 212 into brain 102, tip 504 of drill 502 should penetrate and produce an opening through skull 104 without penetrating through dura mater 114 beneath skull 104. Accordingly, depth stop 506 may be affixed to drill 502 before drilling through scalp 106 such that with tip 504 of drill 502 at surface 108 of scalp 106, distance 510 of bottom 508 of depth stop 506 above top 308 of bushing 302 is less than distance 512 from surface 108 of scalp 106 to dura mater 114. In other words, when drill 502 is spun and pushed down to penetrate scalp 106 and skull 104, depth stop 506 may prevent drill 502 from penetrating dura mater 114.

Depth stop 506 may be preset on drill 502 based upon distance 306 from top 308 of bushing 302 to bottom 304 of bushing 302. As will be further discussed below, distance 512 may be derived based upon an anatomical model of brain 102 co-registered with a scanned image of brain 102 and provided to neuronavigational system 402. After drill 502 has drilled an opening (not shown) through skull 106, drill 502, and the reducing tube if used may be removed from bushing 302. If necessary, coagulation of the drilled area of the scalp 106 and within skull 104 may be executed through the central hollow core of bushing 302 using appropriate instruments with bottom 304 of bushing 302 still in contact with surface 108 of scalp 106 with central hollow core centered around entry point 224.

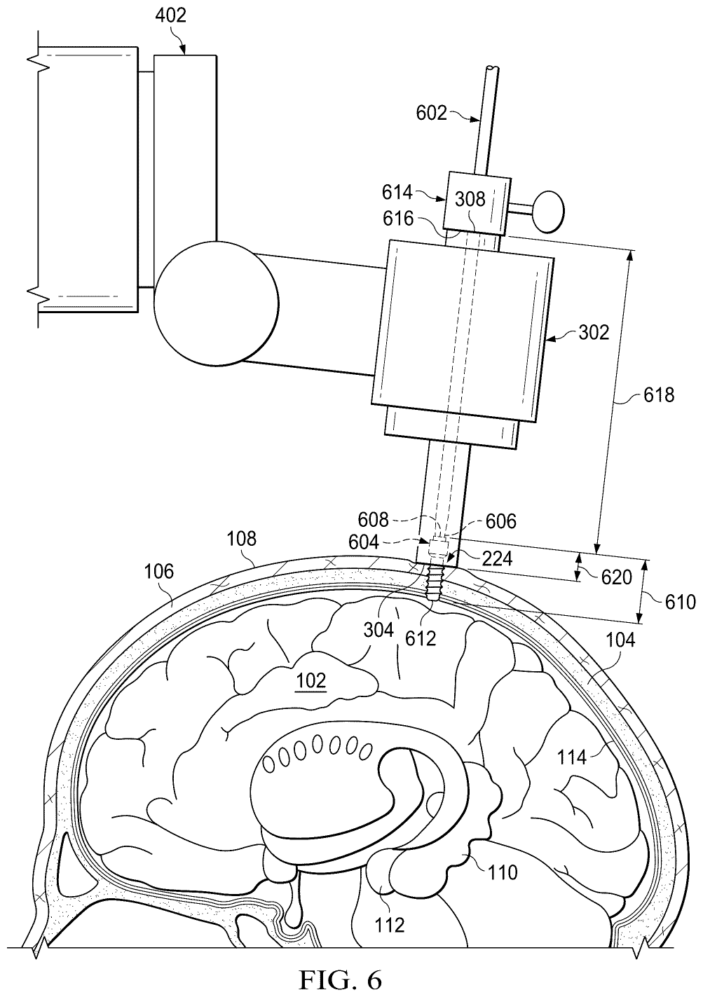

With reference now to FIG. 6, FIG. 6 is an illustration of a cross-sectional side-view of a driver in a bushing of a stereotactic navigation system and an anchor bolt anchored through an opening in a skull in accordance with an illustrative example. More specifically, driver 602 is shown after driving and anchoring anchor bolt 604 into skull 104 with tip 606 of driver 602 still connected to top 608 of anchor bolt 604. As used herein, anchor bolt 604 represents a device configured to attach and/or secure a probe, such as without limitation probe 212, to skull 104. In other words, anchor bolt 604 may be considered and referred to as a fixation device.

Anchor bolt 604 may have length 610 measured from top 608 of anchor bolt 604 to deep end 612 of anchor bolt. Without limitation, length 610 may be one of: 20, 25, 30, or 35 millimeters. Length 610 of anchor bolt 604 may be optimized based upon a thickness of skull 104 and/or a thickness of scalp 106. Anchor bolt 604 may have a diameter of one of, without limitation, 2.1 or 2.4 millimeters, or a diameter appropriate for a diameter of drill 502 used. Depth marker 614 is shown positioned with bottom 616 of depth marker 614 tightened onto driver 602 flush against top 308 of bushing 302.

With anchor bolt 604 anchored in skull 104 before driver 602 is removed from anchor bolt 604 distance 618 exists between bottom of depth marker 614 and tip 606 of driver 602. Distance 618 may be referred to as anchor bolt depth indication (ABDI) 618. ABDI 618 may be measured after driver 602 is removed from anchor bolt 604 and with anchor bolt 604 anchored in skull 104 distance 620 may exist between top 608 of anchor bolt 604 and surface 108 of scalp 106. Distance 620 may be referred to as extension 620 of anchor bolt 604 above surface 108 of scalp 106.

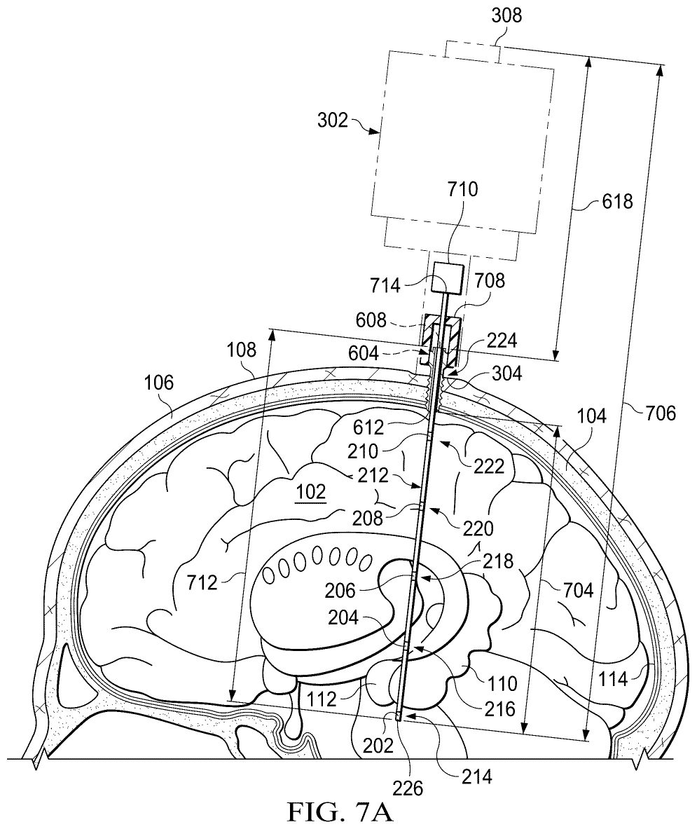

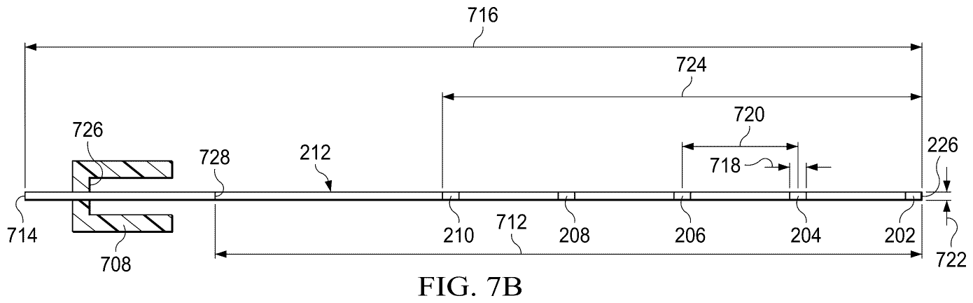

With reference now to FIG. 7A, FIG. 7A is an illustration of a cross-sectional side-view of a portion of a brain with a probe inserted therein and connected to an anchor bolt in accordance with an illustrative example. More specifically, anchor bolt 604 is shown anchored in skull 104 after driver 602 has been removed. Probe 212 is shown inserted in brain 102 such that tip 226 of probe 212 has reached target 214. Cap 708 is set along probe 212 at a location that ensures securing probe 212 at top 608 of anchor bolt 604 in a stable fashion to ensure that tip 226 of probe 212 terminates at target 214. Probe 212 is affixed to cap 708 that is affixed to top 608 of anchor bolt 604.

Therefore, probe 212 has bolted length 712. Bolted length 712 of probe 212 may be measured from tip 226 of probe 212 to top 608 of anchor bolt 604.

Distance 704 shown from deep end 612 of anchor bolt 604 to tip 226 of probe 212 may be referred to as working length 704 of probe 212. Thus, bolted length 712 of probe 212 may be considered to include working length 704 and length 610 for selected anchor bolt 604 anchored in skull 104. Probe 212 may have a number of electrodes (such as but without limitation: electrode 202, electrode 204, electrode 206, electrode 208, and electrode 210) located along working length 704. Without limitation, each electrode may occupy 2.0 millimeters of working length 704 of probe 212. Without limitation, a center of each electrode may be spaced 3.5 to 4.5 millimeters from a center of a nearest electrode.

Also shown in FIG. 7A is the shadow image of bushing 302 at time anchor bolt 604 anchoring in skull 104 is completed. As will be further discussed below, distance 706 exists from top 308 of bushing 302 in the position shown to tip 226 of probe 212 when inserted as desired to reach target 214. Distance 706 may be referred to as target reference length (TRL) 706.

Once anchor bolt 604 is implanted in skull 104, top 308 is set, an actual dimension for distance of ABDI 618 is measured and transmitted to Probe-Select 802. In real time, Probe-Select 802 uses actual dimension for ABDI 618 to instantly derive a desired dimension for working length 704 that will optimize an accuracy of locating tip 226 of probe 212 at target 214 in brain 102. In real time Probe-Select 802 uses real time desired dimension for working length 704 to evaluate and select optimum probe 804 from among probes of inventory 808.

FIG. 7B is an illustration of a cross-sectional side-view of a probe with electrodes for insertion into a brain in accordance with an illustrative example. Specifically, probe 212 shown in FIG. 2 is shown isolated in a zoomed-in cross-sectional side-view.

Probe 212 is shown in FIG. 7B with total length 716 (not shown in FIG. 2). A dimension for total length 716 for most probes currently manufactured is 150 millimeters (mm). Electrode 202, electrode 204, electrode 206, electrode 208, and electrode 210 are shown as rings encircling probe 212. Most probes currently manufactured are produced with width 718 of each electrode on the probe having, respectively, an equal dimension. Without limitation, 0.2 to 1.2 millimeters are common values for width 718 in currently produced probes.

Most probes currently are produced with between 6 to 18 electrodes formed as rings encircling probe 212. For ease of visualization in FIG. 2, FIG. 7B, and FIG. 7A, only five electrodes are shown on probe 212. Most probes currently manufactured are produced with spacing 720 between a midpoint of width 718 of one electrode to a midpoint of width 718 for any adjacent electrode - as shown in FIG. 7B between the nonlimiting illustration of electrode 206 and electrode 208—being equal between all electrodes. In other words, most currently produced probes have a constant spacing 720 between center points of all adjacent electrodes.

Currently manufacturers identify characteristics for probe 212 by, without limitation, dimensions for width 718, diameter 722, number of electrodes and spacing 720, as well as a recording depth 724. Recording depth 724 is the distance from tip 226 of probe 212 with electrode 202 located at tip 226 to the edge of width 718 of farthest most electrode 210 from tip 226. A dimension for recording depth 724 may be varied by changing a dimension for spacing 720 and/or width 718 and or a number of electrodes attached to probe 212.

Thus, recording depth 724 for current probes may be dependent upon spacing 720 and width 718 of electrodes along probe 212. Hence, as a nonlimiting example, for the configuration shown in FIG. 7B, if a dimension of width 718 is 2 millimeters (mm) and a dimension of spacing 720 is 3.5 mm, then recording depth 724 for probe 212 will be 16 mm.

For ease of visualization, probe 212 in FIG. 2 and FIG. 7B is shown with only electrode 202, electrode 204, electrode 206, electrode 208, and electrode 210. More or fewer electrodes may be located along probe 212. Current manufacturers typically place 6 to 18 electrodes along probe 212.

Probe 212 also has diameter 722. Conventional probes currently in production may have, without limitation, a dimension of 1.25 mm for diameter 722.

One of ordinary skill in the art recognizes that one way to overcome the current uncertainty of where probe 212 and electrodes thereon will actually be inside brain 102, and how close electrodes on probe 212 will be to desired targets in brain 102, would be to place as many electrodes as possible with the least spacing possible between them along probe 212. However, more electrodes on probe 212 increases the cost of probe 212. Also, a dimension of diameter 722 may be proportional to a quantity of electrodes located along probe 212. Hence a greater number of electrodes on probe 212 may increase a dimension required for diameter 722.

To minimize invasive impact of probe 212 and minimize rates of complications caused by insertion of probe 212 into brain 102, it is desirable to minimize a dimension for diameter 722. Hence, for at least the reasons above, current manufacture of probes may limit a number of electrodes on probe 212 to 16 or less. Accordingly, a solution is needed to provide for a selection of an optimum probe 804 that is an optimum version of probe 212 that is configured with a number of electrodes with width 718 and spacing 720 that place electrodes closest to desired targeted areas of brain 102.

Probe 212 in FIG. 7B is also shown with cap 708 positioned before being moved down to bolted line 728 before probe 212 is inserted through anchor bolt 604 into brain 102. Cap 708 may be configured to be moved along probe 212, set with bolt seal 726 of cap 708 at bolted line 728, and attached to top 608 of anchor bolt 604 to provide a seal over top 608 of anchor bolt 604. Interior of cap 708 and top 608 of anchor bolt 604 may both be formed to mate and make a secure seal that holds probe 212 in place in brain 102 and prevents entry into brain 102 of any elements from environment above scalp 108. Location of bolted line 728 may be set as a distance from tip 226 of probe 212 as determined by Probe-Select 802 based upon ABDI 618.

One of ordinary skill in the art recognizes that to minimize rates of complications caused by insertion of probe 212 into brain 102, it is beneficial and desirable to minimize a time between placing anchor bolt 604 in skull 104 and securing cap 708 over anchor bolt 604. Accordingly, one of ordinary skill in the art recognizes that to minimize rates of complications caused by insertion of probe 212 into brain 102, it is beneficial and desirable to minimize a time to select, locate, and insert and secure to cap 708 a particular probe 212 with optimum dimensions for characteristics of at least: width 718, spacing 720, total number rings of electrodes, and resultant recording depth 724 for probe 212 that result in the closest alignment possible between electrodes on probe 212 and desired areas of brain 102.

Signal transmitter 710 may be connected to end 714 of probe 212. Without limitation, signal transmitter may be a wire, or a wireless device configured to transmit information and control between probe 212 and electrodes thereon with control unit (not shown) configured to at least communicate with and/or control probe 212 and electrodes thereon.

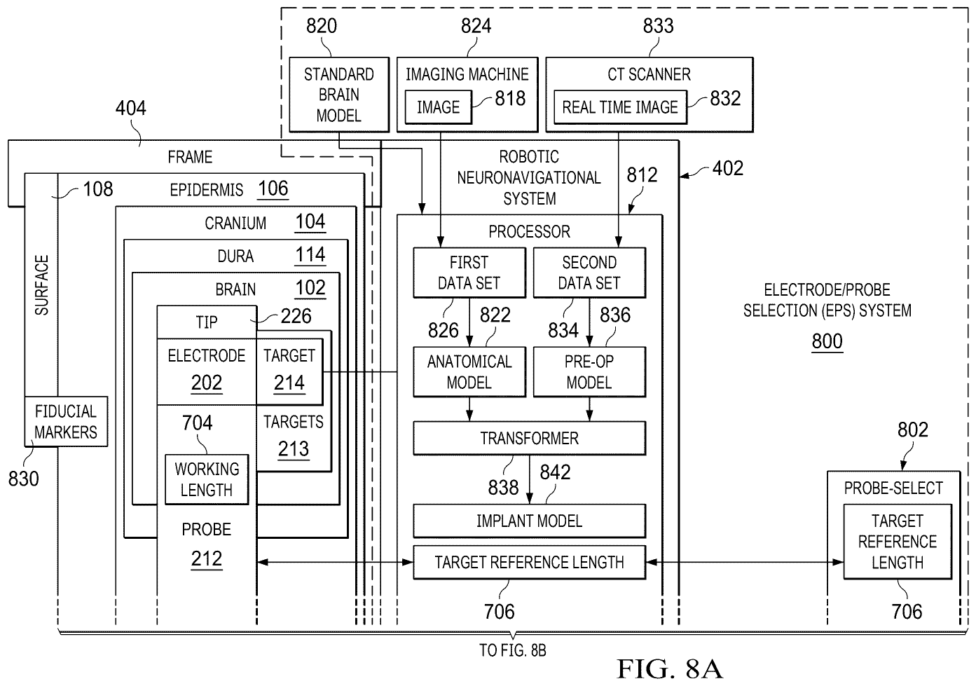

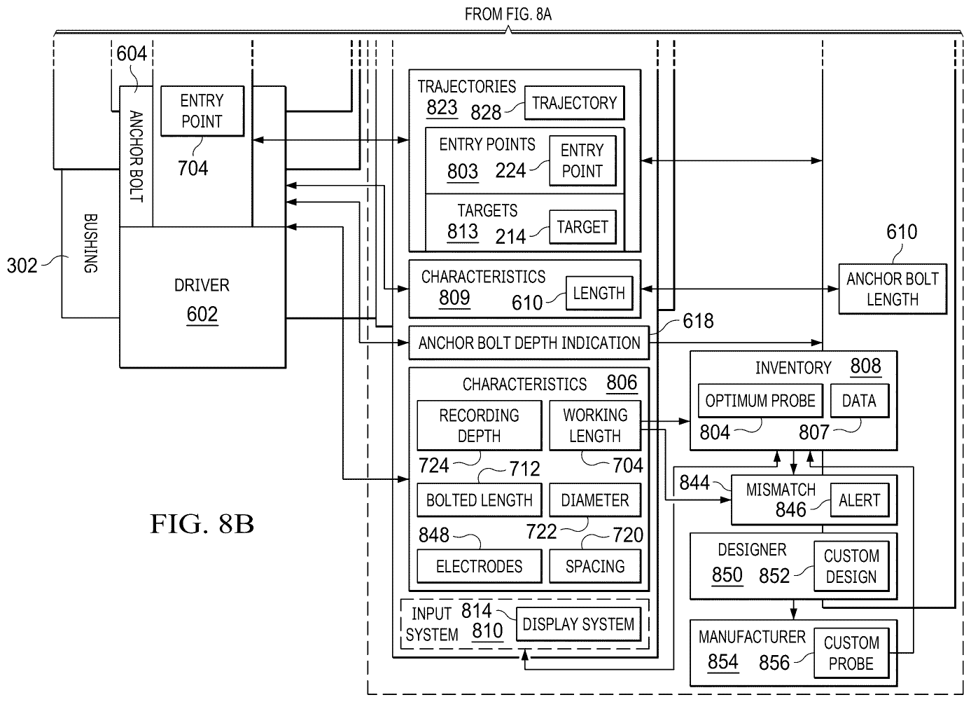

With reference now to FIG. 8A and FIG. 8B, FIG. 8A and FIG. 8B combine to form a block diagram for a machine and process configured to select an optimum probe for insertion into a brain in accordance with an illustrative example. More specifically, FIG. 8A and FIG. 8B illustrate an Electrode or Probe Selection (EPS) System 800 that includes Probe-Select 802. Probe-Select 802 is a formal name given to a processor that is specially programmed to perform EPS that is an executed process of a specially programmed algorithm that selects, in real time, optimum probe 804 for insertion through anchor bolt 604. When a particular probe 212 with specific characteristics 806 is selected by Probe-Select 802 as optimum from among inventory 808 of a number of available probe 212 selections, then probe 212 in the figures above may represent and be referred to as optimum probe 804. Herein, “a number of” is understood by one of ordinary skill in the art to be a whole number that may be one or greater.

In other words, working length 704 may be a length that may be optimized for at least one of recording or stimulation and/or other modulations, such as without limitation: resection, destruction, administration of therapeutics, and/or other functional interaction or therapeutic purpose (heretofore referred to as modulation) of brain 102. In other words, working length 704 may be a length that may be optimized by selecting entry point 224 and target 214 that determine a planned trajectory for probe 212 based upon sites in brain 102 along probe 212 that would be best for modulation of brain 102. This planned working length 704 may direct desired characteristics 806 for a probe 212 that ought to be placed into or ordered to be available in inventory 808 at the time probe 212 is to be inserted into brain 102.

Alternatively, and in real time during insertion of probe 212 into brain 102, once entry point 224 is actually drilled into, and anchor bolt 604 is secured through entry point 224, Probe-Select 802 may select from among probes actually available in inventory 808 which probe 212 therein has characteristics 806 that will produce the optimum modulation of desired areas in brain 102, such as without limitation target 214, target 216, target 218, target 220, and/or target 222. In other words, Probe-Select 802 may provide the technological advantage of determining optimum probe 804 to acquire from suppliers of probes before operation based upon planning for insertion of probe 212 into brain 102, or Probe-Select 802 may provide the technological advantage of determining in real time optimum probe 804 to select out of inventory 808 available during insertion of probe 212 into brain 102. Without limitation, as described further below, Probe-Select 802 may provide the technological advantage of determining custom characteristics when designing a customized optimum probe 804 to be manufactured specifically for a particular insertion into brain 102 at a particular entry point 224 for particular desired targets in brain 102.

Without limitation, Probe-Select 802 can be implemented in a number of different ways. For example, these components can be located in a computer system that may be a physical hardware system that may include one or more data processing systems. When more than one data processing system is present, those data processing systems are in communication with each other using a communications medium. The communications medium may be a network. Without limitation, data processing systems may include at least one of: a head mounted display system, a computer, a server computer, a tablet, or some other suitable data processing system.

Communication with Probe-Select 802 and/or processor 812 may be without limitation, via a physical or wireless communications link. A physical communications link can be established using at least one of a wire cable, an optical cable, or some other physical medium that may allow for an exchange of information between Probe-Select 802 and processor 812. Without limitation, wireless communication can be performed using a wireless link that employs at least one of radiofrequency signals, magnetic signals, or some other type of wireless signal.

Optimum probe 804 may be defined as probe 212 that has characteristics 806 that result in the most precise location of tip 226 of optimum probe 804 at target 214 and any other number of desired electrodes on optimum probe 804 at any other number of designated targets to produce the most precise and/or effective biomedical outcome desired for the monitoring, activations, brain neuromodulation or stimulation for inputs to the brain, and/or any other functions desired for optimum probe 804.

Without limitation, characteristics 806 of optimum probe may include working length 704. As described above and below, the desired functions may include without limitation, monitoring, stimulating, and/or altering a portion of brain 102.

Similarly, anchor bolt 604 in figures above may be selected for insertion through skull 104 with specific characteristics 809 that are received by Probe-Select 802. Characteristics 809 may be without limitation of the skull and the soft tissues along the trajectory of the probe and may include without limitation, length 610 of anchor bolt 604 to be anchored in skull 104, a diameter of anchor bolt 604, material of anchor bolt 604, spacing and thickness of threads on anchor bolt 604, and/or amount and/or type of threads and/or other connection elements near top 608 of anchor bolt 604 configured to retain cap 708. Length 610 and other characteristics of anchor bolt 604 may be selected before drilling into skull, and thus may be received by Probe-Select 802 and/or processor 812 before surgery begins.

If anchor bolt 604 with characteristics 809 alternate from those loaded before surgery is used, the alternate characteristics 809 may be updated through input system 810 connected to Probe-Select 802 and/or processor 812 in real time. Without limitation, input system 810 may be a physical hardware system and may be selected from at least one of: a mouse, a keyboard, a trackball, a touchscreen, a stylus, a motion sensing input machine, a cyber glove, or some other suitable type of input machine.

As described above, numerous technical and medical advantages are generated when optimum probe 804 can be promptly selected in real-time after anchoring anchor bolt 604 into skull 104 and promptly secured thereto as shown in FIG. 7A above.

Probe-Select 802 may be a specially programmed processor that stands alone and communicates with processor 812. Processor 812 may be configured to communicate with and control and may be located within or separated from neuronavigational system 402. Processor 812 may be configured to communicate with Probe-Select 802. Without limitation, processor 812 may represent a number of processors configured to control neuronavigational system 402.

Probe-Select 802 may be integrated with and/or physically located within neuronavigational system 402 or may be integrated with and/or physically located within another neuronavigational device. Probe-Select 802 may be integrated with and/or physically located within processor 812. Probe-Select 802 may receive characteristics 809 from processor 812 or from input system 810 that may be a part of or in communication with Probe-Select 802.

Input system 810 may also communicate with or be a part of neuronavigational system 402. Input system 810 may also communicate with or be a part of another neuronavigational device. Input system 810 may include a number of input units that may be physically connected to or in communication with at least neuronavigational system 402 and/or processor 812 and/or Probe-Select 802. Input system 810 may include a number of input units that may be physically connected to or in communication with another neuronavigational device.

Input system may include display system 814 configured to receive inputs from and/or for at least Probe-Select 802 and processor 812 and thus neuronavigational system 402. Input system may include display system 814 configured to receive inputs from and/or for with another neuronavigational device.

Display system 814 may be further integrated with at least neuronavigational system 402 and/or electrode/probe selection system 800 to provide real time imaging during at least drilling and implantation of probe 212 in brain 102. In other words, display 814 may be configured to provide real time visualization and/or processed depictions of without limitation all objects, lengths, measurements, locations, and derivations related thereto represented in FIG. 8A and FIG. 8B.

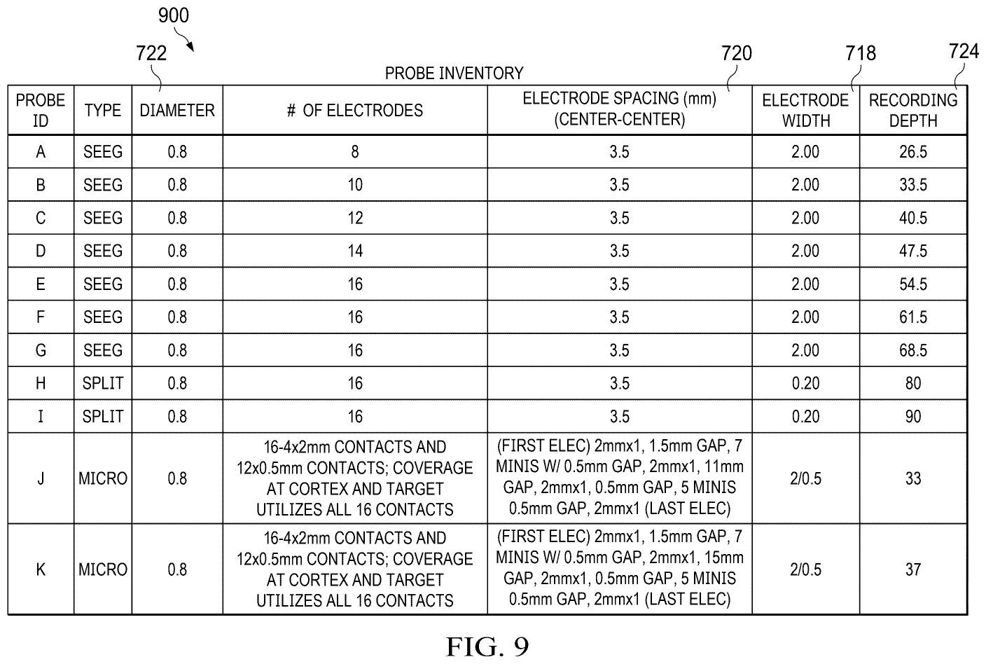

Similarly, display 814 may provide to personnel in operating room presentations such as FIG. 9 of inventory 808, and/or generate all the lengths, measurements, locations, and derivations related thereto represented in FIG. 8A and FIG. 8B in without limitation a chart, table, or graph format. One of ordinary skill in the art recognizes that all presentations on display 814 may also be linked to other displays away from the physical location of brain 102 and personnel operating thereon. Without limitation, display 814 may present a dimensioned diagram of custom design 852 and/or custom probe 856 and/or optimum probe 804. Hence, for at least the reasons presented above, personnel representing a supplier of probes need not be physically present in an operating room as probe 212 is being inserted, nor actively involved in selection of probe 212 at time of implant. Hence, the novel embodiments described herein offer the additional technological advantage of reducing the number of personnel on site as probe 212 is actually being implanted and thereby reducing potential infection, distraction, and/or error possibly caused by having additional personnel on site as probe 212 is actually being implanted.

Without limitation, display system 814 may include a number of displays located in, on, or separated from at least Probe-Select 802 and processor 812 and thus neuronavigational system 402. Display system 814 may be a physical hardware system and may include one or more display machines on which a graphical user interface may be displayed. Without limitation, display machines can include at least one of: a light emitting diode (LED) display, a liquid crystal display (LCD), an organic light emitting diode (OLED) display, or some other suitable machine on which the graphical user interface can be displayed.

Probe-Select 802 also receives from neuronavigational system 402 target reference length (TRL) 706. Without limitation target reference length (TRL) 706 may be derived by neuronavigational system 402. A dimension for target reference length (TRL) 706 may be derived from coordinates for entry point 224 and from coordinates for at least target 214 at desired tip 226 of optimum probe 804 in brain 102. Neuronavigational system 802 may transform entry point 224 and from coordinates for at least target 214 into a dimension for TRL 706.

Distribution and distances between any electrodes along optimum probe 804 for any additional targets other than target 214 at tip 226 of optimum probe 804 may also be received by Probe-Select 802. Probe-Select 802 may receive TDL 706 from processor 812 or from input system 810. Without limitation, input system 810 may be a part of or in communication with Probe-Select 802.

TRL 706 may be derived once coordinates for target 214 and entry point 224 are established. Without limitation, as disclosed at least in U.S. Patent Application Publication 2021/0264623 A1 published Aug. 26, 2021, entitled “Methods for Optimizing the Planning and Placement of Probes in the Brain via Multimodal 3D Analysis of Cerebral Anatomy,” and U.S. Pat. No. 11,497,401 issued Nov. 15, 2022, entitled “Methods for Localization and Visualization of Electrodes and Probes in the Brain Using Anatomical Mesh Models” incorporated herein in its entirety, processor 812 may co-register information in first data set 826 derived from an image 818 taken of brain 102 to form anatomical model 822 for brain 102 designated to receive optimum probe 804. Image 818 of brain 102 may also be referred to as a registration of brain 102. Anatomical model 822 may also include data co-registered from standard brain model 820.

Image 818 of brain 102 may be made by without limitation, imaging machine 824. Imaging machine 824 may utilize without limitation: magnetic resonance imaging sequences (MRI), computerized tomography (CT) sequences, magnetoencephalography (MEG), positron emission tomography (PET), or any combination thereof. Image 818 may represent a number of images. In other words, image 818 may include without limitation, a contrast-weighted MRI scan and a target anatomical MRI scan without contrast.

Image 818 may then be converted from original imaging storage formats into first data set 826 structured with a three-dimensional coordinate system used to form anatomical model 822 of brain 102. Processor 812 may convert image 818 from original imaging storage formats into first data set 826 structured with a three-dimensional coordinate system. Using anatomical model 822, initial coordinates for entry point 224 and at least target 214 at tip 226 of probe 212 may be determined for without limitation, a desired biomedical purpose and/or outcome. Entry point 224 and target 214 being furthermost from entry point 224 define probe trajectory 828 for probe 212. Additional entry points 803 and targets 813 (such as without limitation, target 216, target 218, target 220, and/or target 222) may be established to define additional probe trajectories 823 desired to achieve other desired biomedical purposes and/or outcomes.

Before insertion of any probe 212 into brain 102, fiducial markers 830 may be placed on scalp 106 and a real time scan of brain 102 taken to form real time image (RTI) 832. RTI 832 may also be referred to as a registration of brain 102. Without limitation, real time image 832 may be formed from computerized tomography (CT) sequences taken by CT scanner 833 before drilling begins into skull 104. Real time image 832 may then be converted from original imaging storage formats into second data set 834 structured with a three-dimensional coordinate system used to form pre-op model 836 of brain 102. Processor 812 may convert real time image 832 from original imaging storage formats into second data set 834 structured with a three-dimensional coordinate system.

Processor 812 may then co-register anatomical model 822 with pre-op model 836 through specially programmed algorithm in transformer 838 to produce transformation matrix 840 that aligns first data set 826 with second data set 834 to form implant model 842.

Coordinates from entry point 224 and at least target 214 selected initially in anatomical model 822 are thus updated/transformed to be more precisely defined by implant model 842 and aligned with brain 102 in the operating room for implanting probes. Processor 812 also receives coordinates for precise location and dimension of bushing 302 and moves bushing to locate bottom 304 of bushing 302 against surface 108 of scalp 106 at entry point 224 and therefrom derive and send target reference length 706 to Probe-Select 802.

One of ordinary skill in the art recognizes that for tip 226 of any probe 212 to terminate precisely at target 214, a precise working length 704 of any probe 212 will be dependent upon how far below surface 108 of scalp 106 that deep end 612 of anchor bolt 604 is located. One of ordinary skill in the art recognizes that how far below surface 108 of scalp 106 that deep end 612 of anchor bolt 604 is located may vary based upon characteristics of anchor bolt 604 and an actual location of anchor bolt 604 within skull 104 when anchored in operating room. Once a specified anchor bolt 604 has been selected and implanted in brain 102, characteristics 809 including length 610 of anchor bolt 604 may be received by Probe-Select 802 and/or processor 812 in neuronavigational system 402.

One of ordinary skill in the art recognizes that at time of operation of inserting probe 212 into brain 102, actual scalp 106 may have a thickness or other properties and/or dimensions at entry point 224 than were anticipated based upon standard brain model 820 or as determined pre-operatively by without limitation imaging machine 824 and hence plans made based upon first data set 826 may not be precisely applicable in real time for actual scalp 106 at time that anchor bolt 604 is inserted into skull 104.

Likewise, one of ordinary skill in the art recognizes that at time of operation of inserting probe 212 into brain 102, actual skull 104 may have a thickness or other properties and/or dimensions near entry point 224 than were anticipated based upon standard brain model 820 or as determined pre-operatively by without limitation imaging machine 824 and hence plans made based upon first data set 826 may not be precisely applicable in real time for actual skull 104 at time that anchor bolt 604 is inserted into skull 104. Hence, an actual dimension for working length 704 of probe 212 to target 214 in brain 102 when inserted through anchor bolt 604 may not be equal to a dimension planned for working length 704 of probe 212.

A special program in Probe-Select 802 overcomes in real time the technological problem of inaccuracies from a planned dimension for working length 704, and hence prevents the technological problem of placing electrodes along probe 212 at locations in brain 102 that are not correct to properly modulate desired targets in brain 102. A special program in Probe-Select 802 may detect and hence overcome the technological problem of inaccuracies from a planned dimension for working length 704 by determining in real time an actual dimension for working length 704 when probe 212 is inserted through anchor bolt 604 with cap 708 set at bolted line 728 on probe 212 and cap 708 and thus probe 212 are secured to anchor bolt 604. Further, Probe-Select 802 may also issue alert 846 when actual dimension for working length working length 704 when probe 212 is inserted through anchor bolt 604 with cap 708 set at bolted line 728 on probe 212 and cap 708 and thus probe 212 are secured to anchor bolt 604 differs from a planned dimension for working length 704 when probe 212 is inserted through anchor bolt 604 with cap 708 set at bolted line 728 on probe 212 and cap 708 and thus probe 212 are secured to anchor bolt 604.

When inputs of length 610 of anchor bolt 604, TRL 706, and anchor bolt depth indication (ABDI) 618 are received by Probe-Select 802, specially programmed algorithms in Probe-Select 802 derive a working length 704 desired for probe 212. Probe-Select 802 may also receive coordinates for targets other than target 214. Probe-Select 802 may therefrom derive distances between targets 813, and hence desired spacing 720 between electrodes (such as without limitation: electrode 202, electrode 204, electrode 206, electrode 208, and electrode 210) desired for probe 212, which may be part of characteristics 806 for probe 212.

Probe-Select 802 may also receive and store data 807 for inventory 808 available in operating room for use as probe 212. Probe-Select 802 may also receive and store data 807 configured for use in a design and/or a manufacture of a custom probe and/or electrode(s)based upon a specified trajectory, such as without limitation trajectory 828, for use as probe 212. Trajectory 828 be an optimal trajectory as planned/determined and described above. Data 807 for inventory 808 may include without limitation: number of electrodes on probe 212, type/identification/characteristics/performance of electrodes located on probe 212, distances between electrodes located 704 on probe 212, diameter of probe 212, working length 704 of probe 212, identification/type/model/supplier of probe 212, and bolted length 712 of probe 212.

Without limitation, type of probe 212 may be a micro and/or a split stereo-electroencephalography type. Currently, diameter 722 of probe 212 may be from 0.8 millimeters to 1.2 millimeters may be typical dimensions. Diameter 722 of probe 212 may also be much thinner, including without limitation, in the micron range. Diameter 722 of probe 212 may vary depending on fabrication capacity and use scenario.

Specially programmed algorithms of Probe-Select 802 may process all received inputs to immediately output in real time a selection for optimum probe 804 from among probes available in operating room in inventory 808 that most closely aligns, for actual anchor bolt 604 implanted in skull 104, tip 226 of probe 212 with target 214 as well as other electrodes located on probe 212 with other desired targets (such as without limitation: target 216, target 218, target 220, and/or target 222). Selection of optimum probe 804 may be presented on display system 814 by identifying particular probe 212 from among inventory 808. Identity may include without limitation any and/or all of identification/type/model/supplier/working length 704/number of electrodes, types thereof, and locations on probe 212 selected as optimum probe 804.

Specially programmed algorithms of Probe-Select 802 also immediately derive in real time, mismatch 844 for each probe 212 in inventory 808. Mismatch 844 is a value of a difference between a proposed working length 704 derived from implant model 842 for probe 212 to be optimum probe 804 to be attached to anchor bolt 604 implanted in skull 104 to reach target 214, and actual working length 704 on probe 212 in inventory 808 available for selection as optimum probe 804. As a nonlimiting example, if proposed working length 704 derived from implant model 708 for probe 212 to be optimum probe 804 to be attached to anchor bolt 604 implanted in skull 104 to reach target 214 was 38 mm and actual working length 704 on probe 212 in inventory 808 is 47.3, then value of mismatch 844 is 9.3 mm.

Probe-Select 802 is also configured with special programming configured to (in addition to selecting optimum probe 804 generate alert 846 when mismatch 844 distance equals or exceeds a specified value such as without limitation 10 millimeters or is a negative value. A negative value provides an added technological advantage of recognizing that an electrode along probe 212 may be located within anchor bolt 604. In order for probe 212 to provide useful modulation, no electrode should be within or in contact with anchor bolt 604. Hence, alert 846 from Probe-Select 802 identifies an unacceptable placement of probe that may otherwise not be detected until an attempted use of probe 212 to modulate brain 102. Further, Probe-Select 802 is also configured with special programming configured to (in addition to selecting optimum probe 804 and/or identifying excessive value of mismatch 844) identify in real time when working length 704 derived after anchor bolt 604 is located in skull 104 has a dimension less than recording depth 724, respectively, for any probe 212 in inventory 808 and generate alert 846 respectively therefor.

Probe-Select 802 designates probe 212 in inventory as optimum if it has the lowest positive value of mismatch 844 for all of probes in inventory 212. Probe-Select 802 may also indicate alternate options to optimum probe 804 for any probe 212 in inventory 808 that produce mismatch 844 equal to or less than 9 millimeters. Alert 846 may be presented in display system 814. Alert 846 may be without limitation: aural, visual, tactile and/or transmitted to and/or through some other communication devices.

In other words, Probe-Select 802 is specially programmed to, for any implant model 842 with targets 813, derive mismatch 844 used to select optimum probe 804 from inventory 808 in real time during a procedure immediately after receiving only a value for ABDI 618 after anchor bolt 604 of a known length 610 is implanted in skull 104.

In a similar manner, Probe-Select 802 is specially programmed to design a custom probe 856 in real time during a procedure immediately after receiving only a value for ABDI 618 after anchor bolt 604 of a known length 610 is implanted in skull 104.

Accordingly, Probe-Select 802 is specially programmed to design a custom probe 856 that would provide a pre-operation zero value for mismatch 844 associated with a target value for ABDI 618 to be achieved for implant of custom probe 802.

Characteristics 806 of probe 212 received by Probe-Select 802 may also include number of electrodes 848 on probe 212 and spacing 720 between each of the electrodes 848. Accordingly, measurements and proximities similar to those for target 214 may be applied for locations of each of electrodes 848 along probe 212 in relationship to additional desired targets 813.

Hence, mismatch 844 may be computed not only for working length 704, but for each of electrodes 848 closest thereto, respectively, from among targets 813. Optimum probe selection may then be based upon mismatch value not only for target 214, but for an integrated and/or weighted value of all mismatches for all desired targets, or for some other target designated as primary target among targets 813.

Alternatively, Probe-Select 802 may be loaded with inputs to derive an indication of characteristics needed for probe 212 to be ordered for and/or custom designed to supply inventory 808 prior to operation to implant probe 212 into brain 102. In other words, TRL 706 value input into Probe-Select 802 may be based on coordinates for entry point 224 and at least target 214 in first data set 826—instead of from second data set 834 and/or pre-op model 836 of brain 102—and an anticipated value for ABDI 618 may be received into Probe-Select 802 in order to generate a value for working length 704 that may be used to formulate desired parameters for a particular probe 212 to be fabricated or ordered to stock inventory 808 to closely match anticipated working length 704 generated using second data set 834 and/or pre-op model 836 of brain 102.

Processor 812 and/or Probe-Select 802 can be implemented in software, hardware, firmware or a combination thereof. When software is used, the operations performed by processor 812 and/or Probe-Select 802 can be implemented in program code configured to run on hardware, such as a processor unit. When firmware is used, the operations performed by processor 812 and/or Probe-Select 802 can be implemented in program code and data and stored in persistent memory to run on a processor unit. When hardware is employed, the hardware may include circuits that operate to perform the operations in processor 812 and/or Probe-Select 802. In the illustrative examples, the hardware can take a form selected from at least one of a circuit system, an integrated circuit, an application specific integrated circuit (ASIC), a programmable logic machine, or some other suitable type of hardware configured to perform a number of operations. With a programmable logic machine, the machine may be configured to perform the number of operations. The machine can be reconfigured at a later time or may be permanently configured to perform the number of operations. Programmable logic machines may include, for example, a programmable logic array, a programmable array logic, a field programmable logic array, a field programmable gate array, and other suitable hardware machines.

Additionally, the processes can be implemented in organic components integrated with inorganic components and may be comprised entirely of organic components, excluding a human being. For example, the processes can be implemented as circuits in organic semiconductors.

One or more examples are present that overcome issues with implanting a probe into a brain, and in particular, to more effectively selecting an optimum probe for inserting into a brain for at least stimulation and/or observation. As a result, selection of an optimum probe in real time is provided in a manner that enables an operator, such as a doctor or technician, to manage the operation of implanting a probe into a brain more precisely and with less risk to a patient as compared to operating without the benefits of Probe-Select 802 machine and/or EPS process.

As a result, Probe-Select 802 operates as a special purpose computer system in which inputs are processed to select and/or design an optimum probe for inserting into brain 102 after an anchor bolt 604 is anchored within skull 104 in brain 102. In particular, selection algorithms within Probe-Select 802 render Probe-Select 802 as a special purpose computer system as compared to currently available general computer systems that do not have Probe-Select 802.

The illustration of EPS System 800 in FIG. 8A and FIG. 8B is not meant to imply physical or architectural limitations to the manner in which an illustrative example may be implemented. Other components, in addition to or in place of the ones illustrated, may be used. Some components may be unnecessary. Also, the blocks are presented to illustrate some functional components. One or more of these blocks may be combined, divided, or combined and divided into different blocks when implemented in an illustrative example.

The flowcharts and block diagrams in the different depicted examples illustrate the architecture, functionality, and operation of some possible implementations of apparatuses and methods in an illustrative example. In this regard, each block in the flowcharts or block diagrams can represent at least one of a module, a segment, a function, or a portion of an operation or step. For example, one or more of the blocks can be implemented as program code, hardware, or a combination of the program code and hardware. When implemented in hardware, the hardware can, for example, take the form of integrated circuits that are manufactured or configured to perform one or more operations in the flowcharts or block diagrams. When implemented as a combination of program code and hardware, the implementation can take the form of firmware. Each block in the flowcharts or the block diagrams can be implemented using special purpose hardware systems that perform the different operations or combinations of special purpose hardware and program code run by the special purpose hardware.

In some alternative implementations of an illustrative example, the function or functions noted in the blocks can occur out of the order noted in the figures. For example, in some cases, two blocks shown in succession can be performed substantially concurrently, or the blocks may sometimes be performed in the reverse order, depending upon the functionality involved. Also, other blocks can be added in addition to the illustrated blocks in a flowchart or block diagram.

Turning now to FIG. 9, FIG. 9 is an illustration of an example of an inventory of probes available for implanting into a brain in accordance with an illustrative example. Specifically inventory 808 of probes available for implanting may be formatted as data 807 representing characteristics 806 for probes in inventory 808. Without limitation Probe-Select 802 may be configured with special programming to receive and transform data 807 to decode and process at least the information presented in chart 900 for each respective probe 212 residing within inventory 808.

Currently, spacing 720 between and number of electrodes are set by supplier for any one of probe 212 identified in inventory 808 represented in the nonlimiting example of chart 900. Due to cost of purchasing and/or other supply control reasons, for any given operation for inserting probe 212 into brain 102, only certain combinations of spacing 720 and # of electrodes forming recording depth 724 are usually available in inventory 808.

Chart 900 also indicates that suppliers of probe 212 have varied some probe constructions for special needs with a split type of probe 212. In a split type probe 212, a supplier may form probe 212 with dimension for spacing 720 not being equivalent between every adjacent pair of electrodes on probe 212. As a nonlimiting example, probe 212 identified as H may be intended for an insertion with a segment of probe trajectory 828 passing through white brain matter that does not require any modulation. Hence, having electrodes on portion of probe 212 adjacent to the white brain matter would be a waste of cost and resources and potentially drive an undesirably large diameter 722 for probe 212. Thus, for probe 212 identified as H, spacing between electrodes may be different around gray area of brain 102.

As a nonlimiting example, spacing between and 8^{th} and 9^{th} electrode of split type probe 212 may be much longer (such as without limitation 27.25 mm) instead of a constant 3.5 mm used between all other pairs of adjacent electrodes along probe 212, driving recording depth 724 dimension up to 80 mm or more. Chart 900 also provides an additional example of another split type probe 212 identified as I that has a recording depth extended to 90 mm which may indicate a dimension split spacing 720 between a couple of the 16 electrodes of 37.25 mm instead of the 3.5 mm dimension for spacing 720 between all other pairs of adjacent electrodes along probe 212.

Chart 900 also indicates that suppliers of probe 212 have varied some probe constructions for special needs with micro type of probe 212. In a micro type probe 212, a supplier may form probe 212 with dimension for width 718 not being equivalent between every electrode on probe 212. As a nonlimiting example, probe 212 identified as J indicates that while some electrodes on probe 212 may have a typical dimension of 2 mm for width 718, other electrodes on probe 212 may have a micro dimension of 0.5 mm for their width 718. Dimensions for spacing 720 on micro type probe 212 may also be split. Without limitation dimensions for spacing 720 on micro type probe 212 such as without limitation probes identified as J and K may be equivalent between electrodes with 0.5 mm for their width 718 and different dimensions for spacing 720 between electrodes that have a typical SEEG type dimension of 2 mm for width 718 on probe 212.

Significantly, although suppliers of probe 212 produce various types of probe 212, and inventory 808 might have a mix of all three types of probe 212, currently only configurations set by suppliers are available, and for any insertion of probe 212 into brain 102, only probes in inventory 808 co-located with brain 102 at time of insertion are available. For minimum risk of adverse effects from insertion of probe 212 into brain 102 and to obtain maximum productive results from insertion of probe 212 for modulation of desired targets in brain 102 along probe trajectory 828, a minimum amount of time after inserting anchor bolt 604 into skull 104 and a maximum amount of accuracy is required to select optimum configuration for probe 212 to achieve the alignment of electrodes on probe 212 with desired targets in brain 102 to produce maximum results from insertion of probe 212 for modulation of the desired targets in brain 102 along probe trajectory 828. Transforming at least data 807 form inventory 808 and inputs received from processor 812 representing at least ABDI 618 established at insertion of anchor bolt 604 into brain 102, novel specially programmed algorithms of Probe-Select 802 derive the technological improvement of an optimum correlation in real time between configurations of probes available in inventory 808 the alignment of electrodes on probe 212 with desired targets in brain 102. However, neither suppliers split type nor micro type specialty probes provide a probe 212 that is customized for specific desired targets designated along a desired probe trajectory 828 in a particular brain 102 and created specifically for a particular insertion of the customized probe.

Therefore, Probe-Select 802 may include or be in communication with designer 850 configured to communicate and receive information in processor 812 and therefrom create custom design 852 for probe 212. Custom design 852 derives from targets 813 for a specific individual brain 212 a number of electrodes 848 and for each electrode respectively, width 718 and spacing 720 for and between each one of the number of electrodes 848 designated for placement along probe 212 to generate the most effective modulation of all of targets 813. Thus, custom design 852 may design probe 212 with a minimum number of electrodes required to achieve desired modulation of all of targets 813.

Thus, custom design 852 provides the technological advantage for probe 212 of: a least cost for electrodes, the smallest diameter 722 necessary, and a most effective modulation of all targets 813 as compared to currently supplied probes. Hence, custom design 852 provides the technological advantage for probe 212 of reducing at least invasiveness and risks of medical complications from inserting probe 212 into brain 102.

Custom design 852 may be created well before actual surgery to implant probe 212 into brain. Thus, custom design may be supplied to manufacturer 854 so that custom probe 856 may be manufactured specifically for targets 813 for brain 102 and be delivered to inventory 808 before surgery. Thus, Probe-Select 802 and designer 850 also provide the technological advantage of eliminating the time, potential errors, and risks associated with selecting a probe 212 for inserting into brain 102 after securing anchor bolt 604 in skull 106 as compared to doing so without having custom probe 856 ready for insertion and/or using Probe-Select 802.

Even with custom probe 856 ready for insertion after securing anchor bolt 604 in skull 106, Probe-Select 802 is configured to still instantaneously use ABDI 618 and may generate alert 846 if custom probe 856 characteristics 806 no longer comply with mismatch criteria - or may select one of probes in inventory as optimum probe 804. Processor 812 may be configured to use at least implant model 842 and Probe-Select 802 to identify in real time if coordinates for actual entry point 224 or targets 813 in brain 102 at time of surgery may be different than those used from pre-op model 836 to generate custom design 852 and custom probe 856. Hence, even with a custom probe 856 in inventory, Probe-Select 802 may identify if an optimum probe 804 other than custom probe 856 is in inventory 808 and/or provide alert 846 therefor.

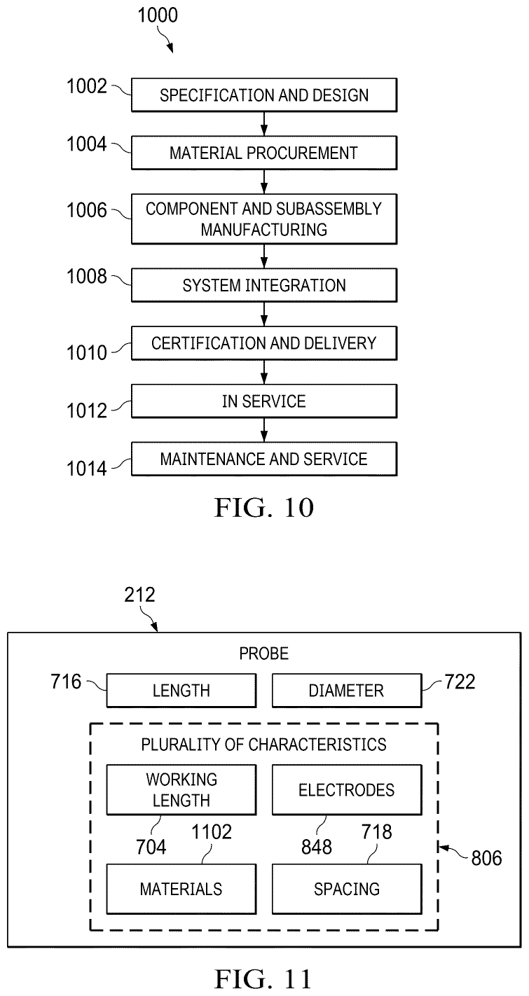

Hence, Illustrative embodiments of the disclosure may be described in the context of probe manufacturing and service process 1000 as shown in FIG. 10 and probe 212 as shown at least in FIG. 2 through FIG. 7B and FIG. 11. Turning first to FIG. 10, an illustration of a probe manufacturing and service method is depicted in accordance with an illustrative embodiment. During pre-production, probe manufacturing and service method 1000 may include specification and design 1002 of probe 212 as described in at least FIG. 2 through FIG. 7B and FIG. 11 and material procurement 1004.

During production, component and subassembly manufacturing 1006 and system integration 1008 of probe 212 in 2 through FIG. 7B and FIG. 11 takes place. Thereafter, probe 212 in FIG. 2 through FIG. 8A and FIG. 11 may go through certification and delivery 1010 in order to be placed in service 1012. While in service 1012 by a customer, probe 212 in FIG. 2 through 8A and FIG. 11 may require routine maintenance and service 1014, which may include without limitation: modification, reconfiguration, refurbishment, and other maintenance or service.

Each of the processes of probe manufacturing and service method 1000 may be performed or carried out by a system integrator, a third party, and/or an operator. In these examples, the operator may be a customer. For the purposes of this description, a system integrator may include, without limitation, any number of probe manufacturers and/or system subcontractors; a third party may include, without limitation, any number of vendors, subcontractors, and suppliers; and an operator may be without limitation: a physician or surgeon or equivalent a medical services provider, a hospital, a health organization, a leasing company, a military entity, and so on.

With reference now to FIG. 11, a block diagram of a probe is depicted in accordance with an illustrative embodiment. In this example, probe 212 may be produced by probe manufacturing and service method 1000 in FIG. 10 and may include total length 716 with plurality of characteristics 806 and electrodes 848. Examples of characteristics 806 may include one or more of diameter 722, working length 704, spacing 718, and materials 1102. Any number of other systems may be included.

Machines and processes embodied herein may be employed during at least one of the stages of probe manufacturing and service method 1000 in FIG. 10.

In one illustrative example, components or subassemblies produced in component and subassembly manufacturing 1006 in FIG. 10 may be fabricated or manufactured in a manner similar to components or subassemblies produced while probe 212 is in service 1012 in FIG. 10. As yet another example, one or more apparatus embodiments, method embodiments, or a combination thereof may be utilized during production stages, such as component and subassembly manufacturing 1006 and system integration 1008 in FIG. 10. One or more apparatus embodiments, method embodiments, or a combination thereof may be utilized while probe 212 is in service 1012 and/or during maintenance and service 1014 in FIG. 10. The use of a number of the different illustrative embodiments may substantially expedite the assembly of and/or reduce the cost of probe 212.

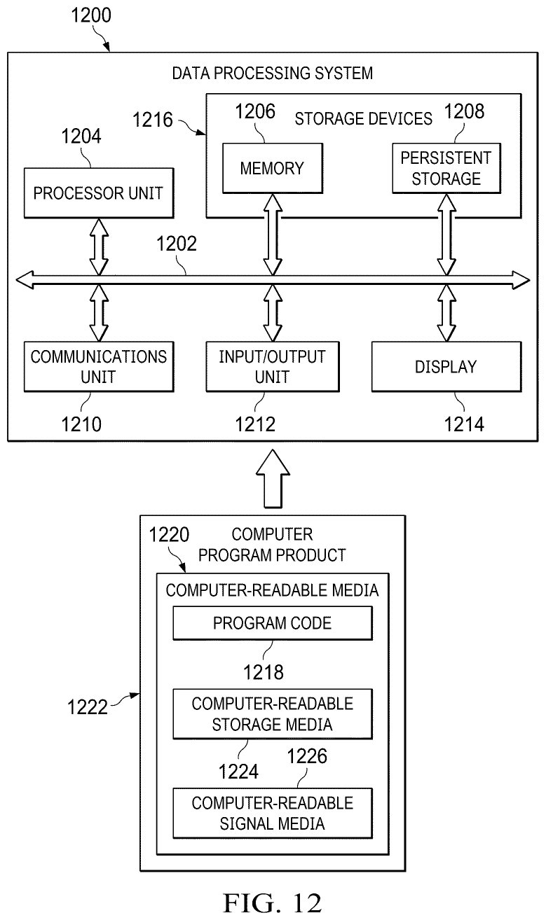

Turning now to FIG. 12, an illustration of a block diagram of a data processing system is depicted in accordance with an illustrative example. Data processing system 1200 may be used to implement processor 812 in neuronavigational system 402 and/or structures and functionalities for Probe-Select 802. In this illustrative example, data processing system 1200 includes communications framework 1202, which provides communications between processor unit 1204, memory 1206, persistent storage 1208, communications unit 1210, input/output unit 1212, and display 1214. In this example, communications framework 1202 can take the form of a bus system.

Processor unit 1204 serves to execute instructions for software that can be loaded into memory 1206. Processor unit 1204 can be a number of processors, a multi-processor core, or some other type of processor, depending on the particular implementation.

Memory 1206 and persistent storage 1208 are examples of storage devices 1216. A storage device may be any piece of hardware that is capable of storing information, such as, for example, without limitation, at least one of data, program code in functional form, or other suitable information either on a temporary basis, a permanent basis, or both on a temporary basis and a permanent basis. Storage devices 1216 can also be referred to as computer-readable storage machines in these illustrative examples. Memory 1206, in these examples, can be, for example, a random-access memory or any other suitable volatile or non-volatile storage machine. Persistent storage 1208 can take various forms, depending on the particular implementation.

For example, persistent storage 1208 may contain one or more components or machines. For example, persistent storage 1208 can be a hard drive, a solid state hard drive, a flash memory, a rewritable optical disk, a rewritable magnetic tape, or some combination of the above. The media used by persistent storage 1208 also can be removable. For example, a removable hard drive can be used for persistent storage 1208.

Communications unit 1210, in these illustrative examples, provides for communications with other data processing systems or machines. In these illustrative examples, communications unit 1210 is a network interface card.

Input/output unit 1212 allows for input and output of data with other machines that can be connected to data processing system 1200. For example, input/output unit 1212 can provide a connection for user input through at least one of a keyboard, a mouse, or some other suitable input machine. Further, input/output unit 1212 can send output to a printer. Display 1214 provides a mechanism to display information to a user. Display 1214 may be a part of or in addition to display system 814 in input system 810 discussed above.

Instructions for at least one of the operating system, applications, or programs may be located in storage devices 1216, which are in communication with processor unit 1204 through communications framework 1202. The processes in the different examples can be performed by processor unit 1204 using computer-implemented instructions, which can be located in a memory, such as memory 1206.

These instructions are referred to as program code, computer usable program code, or computer-readable program code that can be read and executed by a processor in processor unit 1204. The program code in the different examples can be embodied on different physical or computer-readable storage media, such as memory 1206 or persistent storage 1208.

Program code 1218 is located in a functional form on computer-readable media 1220 that is selectively removable and can be loaded onto or transferred to data processing system 1200 for execution by processor unit 1204. Program code 1218 and computer-readable media 1220 form computer program product 1222 in these illustrative examples. In one example, computer-readable media 1220 can be computer-readable storage media 1224 or computer-readable signal media 1226. In these illustrative examples, computer-readable storage media 1224 is a physical or tangible storage machine used to store program code 1218 rather than a medium that propagates or transmits program code 1218.

Alternatively, program code 1218 may be transferred to data processing system 1200 using computer-readable signal media 1226. Computer-readable signal media 1226 can be, for example, a propagated data signal containing program code 1218. For example, computer-readable signal media 1226 can be at least one of an electromagnetic signal, an optical signal, or any other suitable type of signal. These signals can be transmitted over at least one of communications links, such as wireless communications links, optical fiber cable, coaxial cable, a wire, or any other suitable type of communications link.

The different components illustrated for data processing system 1200 are not meant to provide architectural limitations to the manner in which different examples may be implemented. The different illustrative examples can be implemented in a data processing system including components in addition to or in place of those illustrated for data processing system 1200. Other components shown in FIG. 12 can be varied from the illustrative examples shown. The different examples can be implemented using any hardware machine or system capable of running program code 1218.

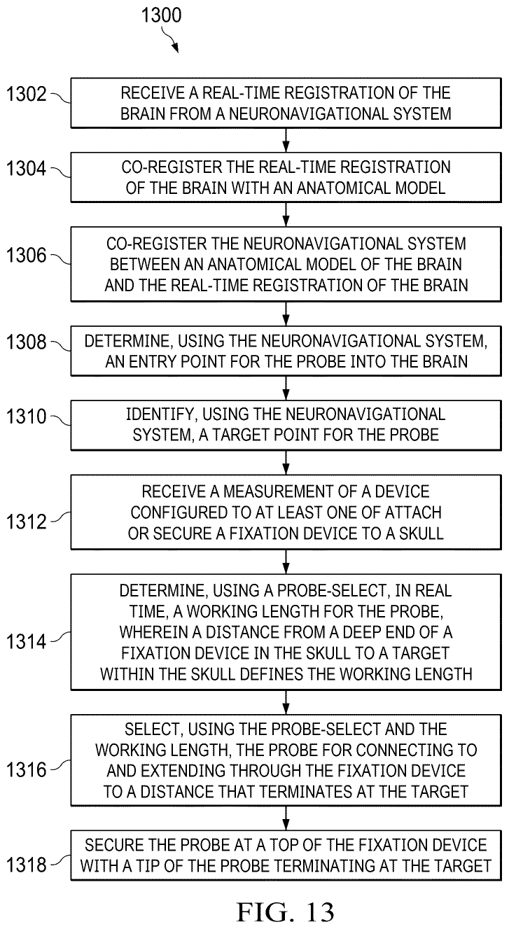

Turning now to FIG. 13, FIG. 13 is an illustration of a flowchart of a process for selecting a probe for inserting into a brain in accordance with an illustrative example. Process 1300 for selecting a probe for inserting into a brain may include at least (operation 1302) receiving a real-time registration of the brain from a neuronavigational system; (operation 1304) co-registering the real-time registration of the brain with an anatomical model; (operation 1306) co-registering the neuronavigational system between an anatomical model of the brain and the real-time registration of the brain; (operation 1308) determining, using the neuronavigational system, an entry point for the probe into the brain; (operation 1310) identifying, using the neuronavigational system, a target point for the probe; (operation 1312) receiving a measurement of a device configured to at least one of attach or secure a fixation device to a skull; (operation 1314) determining, using a Probe-Select, in real time, a working length for the probe, wherein a distance from a deep end of a fixation device in the skull to a target within the skull defines the working length; (operation 1316) selecting, using the Probe-Select and the working length, the probe for connecting to and extending through the fixation device to a distance that terminates at the target; and (operation 1318) securing the probe at a top of the fixation device with a tip of the probe terminating at the target.

Process 1300 for selecting a probe for inserting into a brain may also include at least the working length comprising a length optimized for modulating sites in the brain, located along the probe optimized based on an anatomy, along a length of a planned trajectory of the probe; and the neuronavigational system determining a length from a top of an alignment mechanism of the neuronavigational system to the target. Process 1300 may further include using the anatomical model of the brain for deriving or identifying sites in the brain for optimal electrode implantation for modulation; and determining, using the sites, the entry point and the target defining a planned trajectory for the probe.

Process 1300 may include as well selecting a length of the fixation device optimized for a thickness of an anatomical structure. The probe may include an electrode; and selecting the probe using a location of an electrode located on the probe and a desired site in the brain along the probe may be included in the process as well.

Process 1300 may also include securing the fixation device onto an anatomical structure at the entry point; as well as the probe including functional elements; and one of selecting or manufacturing the probe based upon distances between the functional elements located along the probe.

Further steps of process 1300 may include: the neuronavigational system comprising an alignment mechanism and driving, through the alignment mechanism, the fixation device through the an opening in skull; determining a distance from a surface of a scalp to a top of the fixation device implanted in the skull; attaching a depth stop on a driver connected to the fixation device implanted in the skull; and the Probe-Select receiving and using a distance from the depth stop to a tip of the driver for determining the working length.

Process 1300 may also include: the neuronavigational system comprising an alignment mechanism; connecting a driver to the fixation device and driving the fixation device through the skull; attaching a depth stop on the driver; the Probe-Select receiving a distance from the depth stop to a tip of the driver; and using the distance for determining the working length, wherein the depth stop is set flush at a top of the alignment mechanism after the fixation device is secured in the skull. The probe, including functional elements; and one of selecting or manufacturing the probe based upon distances between electrodes located along the probe, may be steps of process 1300 as well.

Without limitation, process 1300 may also include creating coordinates that define the entry point in a first data set in the anatomical model of the brain in communication with the neuronavigational system; and creating coordinates that define the target in a second data set from a real-time image of the brain co-registered to the first data set in the anatomical model of the brain in communication with the neuronavigational system.

Thus, the illustrative examples provide one or more technical solutions that overcome a technical problem with designing, selecting, and/or implanting a probe into a brain. As a result, one or more technical solutions may provide a technical effect in which a Probe-Select provides a real time design and/or selection for an optimum probe for immediate insertion into a brain.

One or more technical solutions provide a technical benefit that designing, selecting, and/or implanting a probe into a brain may be completed with a more accurate placement of the probe that places electrodes more precisely near a desired target and the exposure time and length of operation are reduced due to immediate automated selection of an optimum probe. Immediate automated selection of an optimum probe also provides the added technical benefit of reducing required number of staff, and thus chances of error and/or infection during and after a procedure.

In this manner, a newer and more intuitive and efficient mechanism is provided to select and optimum probe selection in real time in an operating room for insertion into a brain. The description of the different illustrative examples has been presented for purposes of illustration and description and is not intended to be exhaustive or limited to the examples in the form disclosed. The different illustrative examples describe components that perform actions or operations. In an illustrative example, a component can be configured to perform the action or operation described. For example, the component can have a configuration or design for a structure that provides the component an ability to perform the action or operation that is described in the illustrative examples as being performed by the component.

Many modifications and variations will be apparent to those of ordinary skill in the art. Further, different illustrative examples may provide different features as compared to other desirable examples. The example or examples selected are chosen and described in order to best explain the principles of the examples, the practical application, and to enable others of ordinary skill in the art to understand the disclosure for various examples with various modifications as are suited to the particular use contemplated.

1. A process for selecting a probe for inserting into a brain, the process comprising:
receiving a real-time registration of the brain from a neuronavigational system;
co-registering the real-time registration of the brain with an anatomical model;
co-registering the neuronavigational system between an anatomical model of the brain and the real-time registration of the brain;
determining, using the neuronavigational system, an entry point for the probe into the brain;
identifying, using the neuronavigational system, a target point for the probe;
receiving a measurement of a device configured to at least one of attach or secure a fixation device to a skull;
determining, using a Probe-Select, in real time, a working length for the probe, wherein a distance from a deep end of a fixation device in the skull to a target within the skull defines the working length;
selecting, using the Probe-Select and the working length, the probe for connecting to and extending through the fixation device to a distance that terminates at the target; and
securing the probe at a top of the fixation device with a tip of the probe terminating at the target.

2. The process of claim 1, further comprising:
the working length comprising a length optimized for modulating sites in the brain, located along the probe optimized based on an anatomy, along a length of a planned trajectory of the probe; and
the neuronavigational system determining a length from a top of an alignment mechanism of the neuronavigational system to the target.

3. The process of claim 1, further comprising
using the anatomical model of the brain for deriving or identifying sites in the brain for optimal electrode implantation for modulation; and
determining, using the sites, the entry point and the target defining a planned trajectory for the probe.

4. The process of claim 1, further comprising selecting a length of the fixation device optimized for a thickness of an anatomical structure.

5. The process of claim 1, further comprising:
the probe comprising an electrode; and
selecting the probe using a location of an electrode located on the probe and a desired site in the brain along the probe.

6. The process of claim 1, further comprising:
securing the fixation device onto an anatomical structure at the entry point.

7. The process of claim 1, further comprising:
the probe comprising functional elements; and
one of selecting or manufacturing the probe based upon distances between the functional elements located along the probe.

8. The process of claim 1, further comprising the neuronavigational system comprising an alignment mechanism and driving, through the alignment mechanism, the fixation device through an opening in the skull.

9. The process of claim 1, further comprising determining a distance from a surface of a scalp to a top of the fixation device implanted in the skull.

10. The process of claim 1, further comprising:
attaching a depth stop on a driver connected to the fixation device implanted in the skull; and
the Probe-Select receiving and using a distance from the depth stop to a tip of the driver for determining the working length.

11. The process of claim 1, further comprising:
the neuronavigational system comprising an alignment mechanism;
connecting a driver to the fixation device and driving the fixation device through the skull;
attaching a depth stop on the driver;
the Probe-Select receiving a distance from the depth stop to a tip of the driver; and
using the distance for determining the working length, wherein the depth stop is set flush at a top of the alignment mechanism after the fixation device is secured in the skull.

12. The process of claim 1, further comprising:
the probe comprising functional elements; and
one of selecting or manufacturing the probe based upon distances between electrodes located along the probe.

13. The process of claim 1, further comprising:
creating coordinates that define the entry point in a first data set in the anatomical model of the brain in communication with the neuronavigational system; and
creating coordinates that define the target in a second data set from a real-time image of the brain co-registered to the first data set in the anatomical model of the brain in communication with the neuronavigational system.

14. A machine configured to select an optimum probe to be implanted in a brain, wherein the machine comprises a processor and a non-transitory computer-readable medium that comprises instructions stored thereon and configured to, when executed by a computer system, direct the computer system to:
receive:
an entry point;
a target;
a target reference length;
a length of a fixation device;
a fixation device depth indication; and
an inventory of probes;
derive:
a working length; and
a mismatch between the working length and a proposed working length, respectively, of each probe in the inventory; and
select the optimum probe from the inventory.

15. The machine of claim 14, wherein the processor is further configured to communicate with a second processor in a neuronavigational system that comprises an alignment mechanism.

16. A machine configured to design an optimum probe to be implanted in a brain, wherein the machine comprises a Probe-Select configured to:
select an entry point and a target within the brain;
connect a neuronavigational system to a skull;
select dimensions for a fixation device;
generate a real-time registration of the brain;
co-register the neuronavigational system between an anatomical model of the brain and a pre-op model of the brain;
determine in real time a working length for the optimum probe, wherein a distance from a deep end of the fixation device to the target within the brain defines the working length; and
create, based upon: the working length, the targets, and the entry point, a custom design for the optimum probe that designates: a recoding depth, a number of functional elements, a width for each respective functional element in the number of functional elements, and a spacing respectively between adjacent functional elements, along the optimum probe.

17. The machine of claim 16, wherein the neuronavigational system is further configured to determine a length from a top of an alignment mechanism of the neuronavigational system to the target.

18. The machine of claim 17, wherein:
the Probe-Select is further configured to derive targets that comprise the target in the brain for optimal implantation of the number of functional elements configured to modulate an anatomical model of the brain; and
a depth stop is set flush at a top of the alignment mechanism connected to the fixation device secured in the skull.

19. The machine of claim 16, wherein the Probe-Select is further configured to receive a distance from a depth stop on a driver, used to a tip of the driver connected to the fixation device within the skull.

20. The machine of claim 16, wherein:
the Probe-Select is further configured to receive a number of targets, and
the number of electrodes are located on the optimum probe to modulate the number of targets.
